(19) United States
(12) Patent Application Publication
(10) Pub. No.: US 2017/0107573 A1
(43) Pub. Date: Apr. 20, 2017

(54) METHODS AND SYSTEMS FOR ASSESSING INFERTILITY AS A RESULT OF DECLINING OVARIAN RESERVE AND FUNCTION

(71) Applicant: Celmatix Inc., New York, NY (US)

(72) Inventor: Piraye Yurttas Beim, New York, NY (US)

(21) Appl. No.: 15/298,203

(22) Filed: Oct. 19, 2016

Related U.S. Application Data
(60) Provisional application No. 62/243,659, filed on Oct. 19, 2015.

(51) Int. Cl. C12Q 1/68; A61K 31/138; G06F 19/24; A61K 31/566

(52) U.S. Cl. CPC C12Q 1/6883; A61K 31/566; A61K 31/138; G06F 19/24; C12Q 2600/124; C12Q 2600/158; C12Q 2600/156

(57) ABSTRACT

The present invention relates to methods and systems for assessing ovarian reserve and function in a female subject and informing course of treatment thereof. The invention provides methods for assessing ovarian reserve and function by analyzing both clinical and genetic data/characteristics from a female subject. These methods involve the determination of the presence of one or more mutations in a gene, the gene being associated with fertility and/or ovarian reserve or function. In certain aspects the methods also involve the determination of one or more clinical characteristics associated with fertility and/or ovarian reserve or function. In certain embodiments, the clinical and genetic characteristics obtained from a female subject can be used as data to be input to an ovarian reserve predictor, such that a probability of the female subject suffering from ovarian reserve dysfunction or premature decline can be generated.

### RELATED APPLICATIONS

This application claims the benefit of and priority to U.S. Provisional Patent Application No. 62/243,659, filed Oct. 19, 2015, the content of which is incorporated herein in its entirety.

### BACKGROUND

According to the Centers for Disease Control and Prevention, 6.7 million women (around 10.9%) in the United States between the ages of 15 and 44 suffer from impaired fecundity. See Chandra A, Copen C E, Stephen E H. Infertility and impaired fecundity in the United States, 1982-2010: Data from the National Survey of Family Growth. National health statistics reports; no 67. Hyattsville, Md.: National Center for Health Statistics, 2013. On average, egg quality and number begins to decline precipitously at 35 and reduced fecundity as a result of declining ovarian reserve and function leading up to menopause is a normal part of aging in females. However, in some women, ovarian aging happens prematurely, sometimes resulting in fecundity-related disorders such as diminished ovarian reserve (DOR) or primary ovarian insufficiency (POI).

Primary ovarian insufficiency (POI) refers to the condition wherein a female loses normal function of her ovaries before the age of 40. This loss of function leads to the failure of the ovaries to produce normal amounts of the hormone estrogen. Also stemming from the loss in function is the failure of the ovaries to release eggs on a regular basis. Infertility often results from POI. At the current time, there is no known treatment to restore fertility in females with this condition. One option for pursuing pregnancy for women suffering from infertility from POI is in vitro fertilization (IVF) using donor eggs or eggs that have been harvested and frozen prior to becoming infertile.

Diminished ovarian reserve (DOR) is a condition in which a woman's ovaries contain fewer eggs than would be expected for their age. This can make conception more difficult and decrease the chance of conceiving with IVF and other fertility treatments. DOR can also result in a higher chance of miscarriage compared to miscarriage rates in women not suffering from DOR.

Although genetic studies have shed light on molecular defects associated with DOR and POI, the extent to which they share etiologies was largely unexplored. It was unclear whether POI and DOR are two facets of the same condition with varying levels of severity along the ovarian aging spectrum or two molecularly distinct disorders impacting ovarian function. Our findings suggest the former, that women with shared genetic and clinical risk factors fall along a spectrum, some presenting with DOR and some with POI depending on the severity of the manifestation of those risk factors.

### SUMMARY

The invention relates to methods and systems for assessing risk of premature decline in ovarian reserve and function and informing course of treatment thereof. In some embodiments, the invention provides methods for assessing risk of risk of premature decline in ovarian reserve and function, which includes obtaining sequence reads from sequencing of genomic DNA obtained from a sample, identifying one or more variations in one or more ovarian reserve genes, and characterizing risk of abnormal ovarian reserve and function of the female subject based upon the identification of the one or more variations. Other aspects of the invention involve methods for treating patients experiencing risk of premature decline in ovarian reserve and/or function. The invention also relates to methods for determining key genetic pathways underlying the differences between POI and DOR. The invention also relates to determining key genetic differences underlying severity of the ovarian ageing phenotype along the DOR to POI spectrum.

In one embodiment of the invention, the invention provides a method for assessing risk of premature decline in ovarian reserve or function in a female subject using a computer system comprising a processor coupled to memory. In accordance with the method, the computer system accepts as input, data representative of a plurality of genetic and clinical characteristics of the female subject; analyzes the input data using an ovarian reserve predictor correlated with ovarian reserve and function; and generates a report of the probability of ovarian reserve dysfunction or premature decline in the female subject as a result of using the ovarian reserve predictor on the input data. In one aspect, the ovarian reserve predictor is generated by obtaining reference data from a plurality of females, the reference data corresponding to fertility and/or ovarian reserve-associated genetic and clinical characteristics and diagnoses of ovarian reserve dysfunction or premature decline; and determining one or more correlations between at least one genetic or clinical characteristic and a known diagnosis.

In another embodiment, the invention provides a method for assessing an increased risk of ovarian reserve dysfunction or decline in a female subject, which includes the steps of obtaining a biological sample from the female subject, isolating nucleic acid from said biological sample, performing an assay on the isolated nucleic acid to determine a presence of one or more mutations in a gene, wherein the gene is associated with fertility and/or ovarian reserve or function, and assessing an increased risk of ovarian reserve dysfunction or decline based on the presence of one or more mutations in said gene, where the presence of at least one mutation in said gene is indicative of an increased risk of ovarian reserve dysfunction or decline in said female subject.

In yet another embodiment, the invention provides for a method of treating a female subject suspected of suffering from ovarian dysfunction or decline in ovarian reserve, which includes the steps of conducting an assay to determine a presence of one or more variants in one or more genes associated with infertility and/or ovarian reserve or function, wherein the presence of the one or more variants is indicative that the female subject suffers from a disorder associated with ovarian dysfunction or decline in ovarian reserve; and providing a fertility treatment, including egg freezing, to the female subject based on the indicated disorder.

### BRIEF DESCRIPTION OF DRAWINGS

FIG. 1A illustrates the overlap in affected genes between POI and DOR patients across the entire genome.

FIG. 1B illustrates the overlap in affected genes between POI and DOR patients within ovarian reserve genes.

FIG. 2A illustrates an interaction network between ovarian reserve genes involved in ovarian function and development. Highlighted are gene variants with significantly higher frequency in POI (red) or DOR (blue) compared to the “normal reserve” control group or the general population.

FIG. 2B illustrates an interaction network between ovarian reserve genes involved in DNA repair. Highlighted are gene variants with significantly higher frequency in POI (red) or DOR (blue) compared to the “normal reserve” control group or the general population.

FIG. 3A illustrates a mutation in BMP15's pro-peptide region and how it may alter its dimerization and secretion.

FIG. 3B illustrates BMP15 involvement in the promotion of GC proliferation, regulation of steroidogenesis, and a decrease in GC's responsiveness to FSH.

FIG. 3C illustrates a mutation in the FSHR ligand binding ectodomain that may alter FSHR-FSH interaction.

FIG. 4 gives a diagram of a system of the invention.

FIG. 5 illustrates the regularization path across various penalty parameter values. The dotted line indicates the value of the optimal penalization parameter value as determined by 10-fold cross validation.

### DETAILED DESCRIPTION

The present invention relates to methods and systems for assessing risk of premature decline in ovarian reserve or function in a female subject and informing course of treatment thereof. The invention provides methods for assessing risk of premature decline in ovarian reserve or function by analyzing both clinical and genetic data/characteristics from a female subject. These methods involve the determination of the presence of one or more mutations in a gene, the gene being associated with fertility and/or ovarian reserve or function. In certain aspects the methods also involve the determination of one or more clinical characteristics associated with fertility and/or ovarian reserve or function. In certain embodiments, the clinical and genetic characteristics obtained from a female subject can be used as data to be input to an ovarian reserve predictor, such that a probability of the female subject suffering from ovarian reserve dysfunction or premature decline can be generated.

### Genetic Data

In one aspect of the invention, genetic data includes genetic biomarkers and genetic classifications. These biomarkers and classifications can be utilized to provide more accurate prognoses that can inform downstream diagnostic tests and treatments that may benefit the subject.

Biomarkers for use with methods of the invention may be any marker that is associated with infertility and/or ovarian reserve. Exemplary biomarkers include genes (e.g. any region of DNA encoding a functional product), genetic regions (e.g. regions including genes and intergenic regions with a particular focus on regions conserved throughout evolution in placental mammals), and gene products (e.g., RNA and protein). In certain embodiments, the biomarker is an infertility-associated gene or genetic region. An infertility-associated genetic region is any DNA sequence in which variation is associated with a change in fertility. Examples of changes in fertility include, but are not limited to, the following: a homozygous mutation of an infertility-associated gene leads to a complete loss of fertility; a homozygous mutation of an infertility-associated gene is incompletely penetrant and leads to reduction in fertility that varies from individual to individual; a heterozygous mutation is completely recessive, having no effect on fertility; and the infertility-associated gene is X-linked, such that a potential defect in fertility depends on whether a non-functional allele of the gene is located on an inactive X chromosome (Barr body) or on an expressed X chromosome.

In particular embodiments, the assessed infertility-associated genetic region is a maternal effect gene. Maternal effects genes are genes that have been found to encode key structures and functions in mammalian oocytes (Yurttas et al., Reproduction 139:809-823, 2010). Maternal effect genes are described, for example in, Christians et al. (Mol Cell Biol 17:778-88, 1997); Christians et al., Nature 407:693-694, 2000); Xiao et al. (EMBO J 18:5943-5952, 1999); Tong et al. (Endocrinology 145:1427-1434, 2004); Tong et al. (Nat Genet 26:267-268, 2000); Tong et al. (Endocrinology, 140:3720-3726, 1999); Tong et al. (Hum Reprod 17:903-911, 2002); Ohsugi et al. (Development 135:259-269, 2008); Borowczyk et al. (Proc Natl Acad Sci USA., 2009); and Wu (Hum Reprod 24:415-424, 2009). Maternal effects genes are also described in U.S. Ser. No. 12/889,304. The content of each of these is incorporated by reference herein in its entirety.

In particular embodiments, the infertility-associated genetic region is one or more genes (including exons, introns, and 10 kb of DNA flanking either side of said gene) selected from the genes shown in Table 1 below. In Table 1, OMIM reference numbers are provided when available.

TABLE 1
Human Infertility-Related Genes (OMIM #)
Table 1 - Human Infertility-Related Genes (OMIM #)

| | | | |
|---|---|---|---|
| ABCA1 (600046) | ACTL6A (604958) | ACTL8 | ACVR1 (102576) |
| ACVR1B (601300) | ACVR1C (608981) | ACVR2(102581) | ACVR2A (102581) |
| ACVR2B (602730) | ACVRL1 (601284) | ADA (608958) | ADAMTS1 (605174) |
| ADM (103275) | ADM2 (608682) | AFF2 (300806) | AGT (106150) |
| AHR (600253) | AIRE (607358) | AK2 (103020) | AK7 |
| AKR1C1 (600449) | AKR1C2 (600450) | AKR1C3 (603966) | AKR1C4 (600451) |
| AKT1 (164730) | ALDOA (103850) | ALDOB (612724) | ALDOC (103870) |
| ALPL (171760) | AMBP (176870) | AMD1 (180980) | AMH (600957) |
| AMHR2 (600956) | ANK3 (600465) | ANXA1 (151690) | APC (611731) |
| APOA1 (107680) | APOE (107741) | AQP4 (600308) | AR (313700) |
| AREG (104640) | ARF1 (103180) | ARF3 (103190) | ARF4 (601177) |
| ARF5 (103188) | ARFRP1 (604699) | ARL1 (603425) | ARL10 (612405) |
| ARL11 (609351) | ARL13A | ARL13B (608922) | ARL15 |
| ARL2 (601175) | ARL3 (604695) | ARL4A (604786) | ARL4C (604787) |
| ARL4D (600732) | ARL5A (608960) | ARL5B (608909) | ARL5C |
| ARL6 (608845) | ARL8A | ARL8B | ARMC2 |
| ARNTL (602550) | ASCL2 (601886) | ATF7IP (613644) | ATG7 (608760) |
| ATM (607585) | ATR (601215) | ATXN2 (601517) | AURKA (603072) |
| AURKB (604970) | AUTS2 (607270) | BARD1 (601593) | BAX (600040) |
| BBS1 (209901) | BBS10 (610148) | BBS12 (610683) | BBS2 (606151) |
| BBS4 (600374) | BBS5 (603650) | BBS7 (607590) | BBS9 (607968) |
| BCL2 (151430) | BCL2L1 (600039) | BCL2L10 (606910) | BDNF (113505) |
| BECN1 (604378) | BHMT (602888) | BLVRB (600941) | BMP15 (300247) |
| BMP2 (112261) | BMP3 (112263) | BMP4 (112262) | BMP5 (112265) |
| BMP6 (112266) | BMP7 (112267) | BMPR1A (601299) | BMPR1B (603248) |
| BMPR2 (600799) | BNC1 (601930) | BOP1 (610596) | BRCA1 (113705) |
| BRCA2 (600185) | BRIP1 (605882) | BRSK1 (609235) | BRWD1 |
| BSG (109480) | BTG4 (605673) | BUB1 (602452) | BUB1B (602860) |
| C2orf86 (613580) | C3 (120700) | C3orf56 | C6orf221 (611687) |
| CA1 (114800) | CARD8 (609051) | CARM1 (603934) | CASP1 (147678) |
| CASP2 (600639) | CASP5 (602665) | CASP6 (601532) | CASP8 (601763) |
| CBS (613381) | CBX1 (604511) | CBX2 (602770) | CBX5 (604478) |
| CCDC101 (613374) | CCDC28B (610162) | CCL13 (601391) | CCL14 (601392) |
| CCL4 (182284) | CCL5 (187011) | CCL8 (602283) | CCND1 (168461) |
| CCND2 (123833) | CCND3 (123834) | CCNH (601953) | CCS (603864) |
| CD19 (107265) | CD24 (600074) | CD55 (125240) | CD81 (186845) |
| CD9 (143030) | CDC42 (116952) | CDK4 (123829) | CDK6 (603368) |
| CDK7 (601955) | CDKN1B (600778) | CDKN1C (600856) | CDKN2A (600160) |
| CDX2 (600297) | CDX4 (300025) | CEACAM20 | CEBPA (116897) |
| CEBPB (189965) | CEBPD (116898) | CEBPE (600749) | CEBPG (138972) |
| CEBPZ (612828) | CELF1 (601074) | CELF4 (612679) | CENPB (117140) |
| CENPF (600236) | CENPI (300065) | CEP290 (610142) | CFC1 (605194) |
| CGA (118850) | CGB (118860) | CGB1 (608823) | CGB2 (608824) |
| CGB5 (608825) | CHD7 (608892) | CHST2 (603798) | CLDN3 (602910) |
| COIL (600272) | COL1A2 (120160) | COL4A3BP | COMT (116790) |
| | | (604677) | |
| COPE (606942) | COX2 (600262) | CP (117700) | CPEB1 (607342) |
| CRHR1 (122561) | CRYBB2 (123620) | CSF1 (120420) | CSF2 (138960) |
| CSTF1 (600369) | CSTF2 (600368) | CTCF (604167) | CTCFL (607022) |
| CTF2P | CTGF (121009) | CTH (607657) | CTNNB1 (116806) |
| CUL1 (603134) | CX3CL1 (601880) | CXCL10 (147310) | CXCL9 (601704) |
| CXorf67 | CYP11A1 (118485) | CYP11B1 (610613) | CYP11B2 (124080) |
| CYP17A1 (609300) | CYP19A1 (107910) | CYP1A1 (108330) | CYP27B1 (609506) |
| DAZ2 (400026) | DAZL (601486) | DCTPP1 | DDIT3 (126337) |
| DDX11 (601150) | DDX20 (606168) | DDX3X (300160) | DDX43 (606286) |
| DEPDC7 (612294) | DHFR (126060) | DHFRL1 | DIAPH2 (300108) |
| DICER1 (606241) | DKK1 (605189) | DLC1 (604258) | DLGAP5 |
| DMAP1 (605077) | DMC1 (602721) | DNAJB1 (604572) | DNMT1 (126375) |
| DNMT3B (602900) | DPPA3 (608408) | DPPA5 (611111) | DPYD (612779) |
| DTNBP1 (607145) | DYNLL1 (601562) | ECHS1 (602292) | EEF1A1 (130590) |
| EEF1A2 (602959) | EFNA1 (191164) | EFNA2 (602756) | EFNA3 (601381) |
| EFNA4 (601380) | EFNA5 (601535) | EFNB1 (300035) | EFNB2 (600527) |
| EFNB3 (602297) | EGR1 (128990) | EGR2 (129010) | EGR3 (602419) |
| EGR4 (128992) | EHMT1 (607001) | EHMT2 (604599) | EIF2B2 (606454) |
| EIF2B4 (606687) | EIF2B5 (603945) | EIF2C2 (606229) | EIF3C (603916) |
| EIF3CL (603916) | EPHA1 (179610) | EPHA10 (611123) | EPHA2 (176946) |
| EPHA3 (179611) | EPHA4 (602188) | EPHA5 (600004) | EPHA6 (600066) |
| EPHA7 (602190) | EPHA8 (176945) | EPHB1 (600600) | EPHB2 (600997) |
| EPHB3 (601839) | EPHB4 (600011) | EPHB6 (602757) | ERCC1 (126380) |
| ERCC2 (126340) | EREG (602061) | ESR1 (133430) | ESR2 (601663) |
| ESR2 (601663) | ESRRB (602167) | ETV5 (601600) | EZH2 (601573) |
| EZR (123900) | FANCC (613899) | FANCG (602956) | FANCL (608111) |
| FAR1 | FAR2 | FASLG (134638) | FBN1 (134797) |
| FBN2 (612570) | FBN3 (608529) | FBRS (608601) | FBRSL1 |
| FBXO10 (609092) | FBXO11 (607871) | FCRL3 (606510) | FDXR (103270) |
| FGF23 (605380) | FGF8 (600483) | FGFBP1 (607737) | FGFBP3 |
| FGFR1 (136350) | FHL2 (602633) | FIGLA (608697) | FILIP1L (612993) |
| FKBP4 (600611) | FMN2 (606373) | FMR1 (309550) | FOLR1 (136430) |
| FOLR2 (136425) | FOXE1 (602617) | FOXL2 (605597) | FOXN1 (600838) |
| FOXO3 (602681) | FOXP3 (300292) | FRZB (605083) | FSHB (136530) |
| FSHR (136435) | FST (136470) | GALT (606999) | GBP5 (611467) |
| GCK (138079) | GDF1 (602880) | GDF3 (606522) | GDF9 (601918) |
| GGT1 (612346) | GJA1 (121014) | GJA10 (611924) | GJA3 (121015) |
| GJA4 (121012) | GJA5 (121013) | GJA8 (600897) | GJB1 (304040) |
| GJB2 (121011) | GJB3 (603324) | GJB4 (605425) | GJB6 (604418) |
| GJB7 (611921) | GJC1 (608655) | GJC2 (608803) | GJC3 (611925) |
| GJD2 (607058) | GJD3 (607425) | GJD4 (611922) | GNA13 (604406) |
| GNB2 (139390) | GNRH1 (152760) | GNRH2 (602352) | GNRHR (138850) |
| GPC3 (300037) | GPRC5A (604138) | GPRC5B (605948) | GREM2 (608832) |
| GRN (138945) | GSPT1 (139259) | GSTA1 (138359) | H19 (103280) |
| H1FOO (142709) | HABP2 (603924) | HADHA (600890) | HAND2 (602407) |
| HBA1 (141800) | HBA2 (141850) | HBB (141900) | HELLS (603946) |
| HK3 (142570) | HMOX1 (141250) | HNRNPK (600712) | HOXA11 (142958) |
| HPGD (601688) | HS6ST1 (604846) | HSD17B1 (109684) | HSD17B12 (609574) |
| HSD17B2 (109685) | HSD17B4 (601860) | HSD17B7 (606756) | HSD3B1 (109715) |
| HSF1 (140580) | HSF2BP (604554) | HSP90B1 (191175) | HSPG2 (142461) |
| HTATIP2 (605628) | ICAM1 (147840) | ICAM2 (146630) | ICAM3 (146631) |
| IDH1 (147700) | IFI30 (604664) | IFITM1 (604456) | IGF1 (147440) |
| IGF1R (147370) | IGF2 (147470) | IGF2BP1 (608288) | IGF2BP2 (608289) |
| IGF2BP3 (608259) | IGF2BP3 (608259) | IGF2R (147280) | IGFALS (601489) |
| IGFBP1 (146730) | IGFBP2 (146731) | IGFBP3 (146732) | IGFBP4 (146733) |
| IGFBP5 (146734) | IGFBP6 (146735) | IGFBP7 (602867) | IGFBPL1 (610413) |
| IL10 (124092) | IL11RA (600939) | IL12A (161560) | IL12B (161561) |
| IL13 (147683) | IL17A (603149) | IL17B (604627) | IL17C (604628) |
| IL17D (607587) | IL17F (606496) | IL1A (147760) | IL1B (147720) |
| IL23A (605580) | IL23R (607562) | IL4 (147780) | IL5 (147850) |
| IL5RA (147851) | IL6 (147620) | IL6ST (600694) | IL8 (146930) |
| ILK (602366) | INHA (147380) | INHBA (147290) | INHBB (147390) |
| IRF1 (147575) | ISG15 (147571) | ITGA11 (604789) | ITGA2 (192974) |
| ITGA3 (605025) | ITGA4 (192975) | ITGA7 (600536) | ITGA9 (603963) |
| ITGAV (193210) | ITGB1 (135630) | JAG1 (601920) | JAG2 (602570) |
| JARID2 (601594) | JMY (604279) | KAL1 (300836) | KDM1A (609132) |
| KDM1B (613081) | KDM3A (611512) | KDM4A (609764) | KDM5A (180202) |
| KDM5B (605393) | KHDC1 (611688) | KIAA0430 (614593) | KIF2C (604538) |
| KISS1 (603286) | KISS1R (604161) | KITLG (184745) | KL (604824) |
| KLF4 (602253) | KLF9 (602902) | KLHL7 (611119) | LAMC1 (150290) |
| LAMC2 (150292) | LAMP1 (153330) | LAMP2 (309060) | LAMP3 (605883) |
| LDB3 (605906) | LEP (164160) | LEPR (601007) | LFNG (602576) |
| LHB (152780) | LHCGR (152790) | LHX8 (604425) | LIF (159540) |
| LIFR (151443) | LIMS1 (602567) | LIMS2 (607908) | LIMS3 |
| LIMS3L | LIN28 (611043) | LIN28B (611044) | LMNA (150330) |
| LOC613037 | LOXL4 (607318) | LPP (600700) | LYRM1 (614709) |
| MAD1L1 (602686) | MAD2L1 (601467) | MAD2L1BP | MAF (177075) |
| MAP3K1 (600982) | MAP3K2 (609487) | MAPK1 (176948) | MAPK3 (601795) |
| MAPK8 (601158) | MAPK9 (602896) | MB21D1 (613973) | MBD1 (156535) |
| MBD2 (603547) | MBD3 (603573) | MBD4 (603574) | MCL1 (159552) |
| MCM8 (608187) | MDK (162096) | MDM2 (164785) | MDM4 (602704) |
| MECP2 (300005) | MED12 (300188) | MERTK (604705) | METTL3 (612472) |
| MGAT1 (160995) | MITF (156845) | MKKS (604896) | MKS1 (609883) |
| MLH1 (120436) | MLH3 (604395) | MOS (190060) | MPPED2 (600911) |
| MRS2 | MSH2 (609309) | MSH3 (600887) | MSH4 (602105) |
| MSH5 (603382) | MSH6 (600678) | MST1 (142408) | MSX1 (142983) |
| MSX2 (123101) | MTA2 (603947) | MTHFD1 (172460) | MTHFR (607093) |
| MTO1 (614667) | MTOR (601231) | MTRR (602568) | MUC4 (158372) |
| MVP (605088) | MX1 (147150) | MYC (190080) | NAB1 (600800) |
| NAB2 (602381) | NAT1 (108345) | NCAM1 (116930) | NCOA2 (601993) |
| NCOR1 (600849) | NCOR2 (600848) | NDP (300658) | NFE2L3 (604135) |
| NLRP1 (606636) | NLRP10 (609662) | NLRP11 (609664) | NLRP12 (609648) |
| NLRP13 (609660) | NLRP14 (609665) | NLRP2 (609364) | NLRP3 (606416) |
| NLRP4 (609645) | NLRP5 (609658) | NLRP6 (609650) | NLRP7 (609661) |
| NLRP8 (609659) | NLRP9 (609663) | NNMT (600008) | NOBOX (610934) |
| NODAL (601265) | NOG (602991) | NOS3 (163729) | NOTCH1 (190198) |
| NOTCH2 (600275) | NPM2 (608073) | NPR2 (108961) | NR2C2 (601426) |
| NR3C1 (138040) | NR5A1 (184757) | NR5A2 (604453) | NRIP1 (602490) |
| NRIP2 | NRIP3 (613125) | NTF4 (162662) | NTRK1 (191315) |
| NTRK2 (600456) | NUPR1 (614812) | OAS1 (164350) | OAT (613349) |
| OFD1 (300170) | OOEP (611689) | ORAI1 (610277) | OTC (300461) |
| PADI1 (607934) | PADI2 (607935) | PADI3 (606755) | PADI4 (605347) |
| PADI6 (610363) | PAEP (173310) | PAIP1 (605184) | PARP12 (612481) |
| PCNA (176740) | PCP4L1 | PDE3A (123805) | PDK1 (602524) |
| PGK1 (311800) | PGR (607311) | PGRMC1 (300435) | PGRMC2 (607735) |
| PIGA (311770) | PIM1 (164960) | PLA2G2A (172411) | PLA2G4C (603602) |
| PLA2G7 (601690) | PLAC1L | PLAG1 (603026) | PLAGL1 (603044) |
| PLCB1 (607120) | PMS1 (600258) | PMS2 (600259) | POF1B (300603) |
| POLG (174763) | POLR3A (614258) | POMZP3 (600587) | POU5F1 (164177) |
| PPID (601753) | PPP2CB (176916) | PRDM1 (603423) | PRDM9 (609760) |
| PRKCA (176960) | PRKCB (176970) | PRKCD (176977) | PRKCDBP |
| PRKCE (176975) | PRKCG (176980) | PRKCQ (600448) | PRKRA (603424) |
| PRLR (176761) | PRMT1 (602950) | PRMT10 (307150) | PRMT2 (601961) |
| PRMT3 (603190) | PRMT5 (604045) | PRMT6 (608274) | PRMT7 (610087) |
| PRMT8 (610086) | PROK1 (606233) | PROK2 (607002) | PROKR1 (607122) |
| PROKR2 (607123) | PSEN1 (104311) | PSEN2 (600759) | PTGDR (604687) |
| PTGER1 (176802) | PTGER2 (176804) | PTGER3 (176806) | PTGER4 (601586) |
| PTGES (605172) | PTGES2 (608152) | PTGES3 (607061) | PTGFR (600563) |
| PTGFRN (601204) | PTGS1 (176805) | PTGS2 (600262) | PTN (162095) |
| PTX3 (602492) | QDPR (612676) | RAD17 (603139) | RAX (601881) |
| RBP4 (180250) | RCOR1 (607675) | RCOR2 | RCOR3 |
| RDH11 (607849) | REC8 (608193) | REXO1 (609614) | REXO2 (607149) |
| RFPL4A (612601) | RGS2 (600861) | RGS3 (602189) | RSPO1 (609595) |
| RTEL1 (608833) | SAFB (602895) | SAR1A (607691) | SAR1B (607690) |
| SCARB1 (601040) | SDC3 (186357) | SELL (153240) | SEPHS1 (600902) |
| SEPHS2 (606218) | SERPINA10 | SFRP1 (604156) | SFRP2 (604157) |
| | (605271) | | |
| SFRP4 (606570) | SFRP5 (604158) | SGK1 (602958) | SGOL2 (612425) |
| SH2B1 (608937) | SH2B2 (605300) | SH2B3 (605093) | SIRT1 (604479) |
| SIRT2 (604480) | SIRT3 (604481) | SIRT4 (604482) | SIRT5 (604483) |
| SIRT6 (606211) | SIRT7 (606212) | SLC19A1 (600424) | SLC28A1 (606207) |
| SLC28A2 (606208) | SLC28A3 (608269) | SLC2A8 (605245) | SLC6A2 (163970) |
| SLC6A4 (182138) | SLCO2A1 (601460) | SLITRK4 (300562) | SMAD1 (601595) |
| SMAD2 (601366) | SMAD3 (603109) | SMAD4 (600993) | SMAD5 (603110) |
| SMAD6 (602931) | SMAD7 (602932) | SMAD9 (603295) | SMARCA4 (603254) |
| SMARCA5 (603375) | SMC1A (300040) | SMC1B (608685) | SMC3 (606062) |
| SMC4 (605575) | SMPD1 (607608) | SOCS1 (603597) | SOD1 (147450) |
| SOD2 (147460) | SOD3 (185490) | SOX17 (610928) | SOX3 (313430) |
| SPAG17 | SPARC (182120) | SPIN1 (609936) | SPN (182160) |
| SPO11 (605114) | SPP1 (166490) | SPSB2 (611658) | SPTB (182870) |
| SPTBN1 (182790) | SPTBN4 (606214) | SRCAP (611421) | SRD5A1 (184753) |
| SRSF4 (601940) | SRSF7 (600572) | ST5 (140750) | STAG3 (608489) |
| STAR (600617) | STARD10 | STARD13 (609866) | STARD3 (607048) |
| STARD3NL | STARD4 (607049) | STARD5 (607050) | STARD6 (607051) |
| (611759) | | | |
| STARD7 | STARD8 (300689) | STARD9 (614642) | STAT1 (600555) |
| STAT2 (600556) | STAT3 (102582) | STAT4 (600558) | STAT5A (601511) |
| STAT5B (604260) | STAT6 (601512) | STC1 (601185) | STIM1 (605921) |
| STK3 (605030) | SULT1E1 (600043) | SUZ12 (606245) | SYCE1 (611486) |
| SYCE2 (611487) | SYCP1 (602162) | SYCP2 (604105) | SYCP3 (604759) |
| SYNE1 (608441) | SYNE2 (608442) | TAC3 (162330) | TACC3 (605303) |
| TACR3 (162332) | TAF10 (600475) | TAF3 (606576) | TAF4 (601796) |
| TAF4B (601689) | TAF5 (601787) | TAF5L | TAF8 (609514) |
| TAF9 (600822) | TAP1 (170260) | TBL1X (300196) | TBXA2R (188070) |
| TCL1A (186960) | TCL1B (603769) | TCL6 (604412) | TCN2 (613441) |
| TDGF1 (187395) | TERC (602322) | TERF1 (600951) | TERT (187270) |
| TEX12 (605791) | TEX9 | TF (190000) | TFAP2C (601602) |
| TFPI (152310) | TFPI2 (600033) | TG (188450) | TGFB1 (190180) |
| TGFB1I1 (602353) | TGFBR3 (600742) | THOC5 (612733) | THSD7B |
| TLE6 (612399) | TM4SF1 (191155) | TMEM67 (609884) | TNF (191160) |
| TNFAIP6 (600410) | TNFSF13B (603969) | TOP2A (126430) | TOP2B (126431) |
| TP53 (191170) | TP53I3 (605171) | TP63 (603273) | TP73 (601990) |
| TPMT (187680) | TPRXL (611167) | TPT1 (600763) | TRIM32 (602290) |
| TSC2 (191092) | TSHB (188540) | TSIX (300181) | TTC8 (608132) |
| TUBB4Q (158900) | TUFM (602389) | TYMS (188350) | UBB (191339) |
| UBC (191340) | UBD (606050) | UBE2D3 (602963) | UBE3A (601623) |
| UBL4A (312070) | UBL4B (611127) | UIMC1 (609433) | UQCR11 (609711) |
| UQCRC2 (191329) | USP9X (300072) | VDR (601769) | VEGFA (192240) |
| VEGFB (601398) | VEGFC (601528) | VHL (608537) | VIM (193060) |
| VKORC1 (608547) | VKORC1L1 | WAS (300392) | WISP2 (603399) |
| | (608838) | | |
| WNT7A (601570) | WNT7B (601967) | WT1 (607102) | XDH (607633) |
| XIST (314670) | YBX1 (154030) | YBX2 (611447) | ZAR1 (607520) |
| ZFX (314980) | ZNF22 (194529) | ZNF267 (604752) | ZNF689 |
| ZNF720 | ZNF787 | ZNF84 | ZP1 (195000) |
| ZP2 (182888) | ZP3 (182889) | ZP4 (613514) | |

The genes listed in Table 1 can be involved in different aspects of reproduction/fertility related processes. It is also to be understood that additional genes beyond those maternal effect genes listed in Table 1 can also affect fertility.

Biomarkers according to the invention also include genes involved with a number of biological processes, or functional biological classifications, such as processes, or classifications, related to the reproductive process, ovarian function and development, response to hormonal stimulation, oogenesis, regulation of apoptosis, regulation of transcription, cell cycle process, and DNA repair, many of which are listed above in Table 1. Variants in genes associated with these various processes result in fertility difficulties for individuals whose DNA contains these variants and are evidence of ovarian dysfunction and/or premature decline in ovarian reserve, as well as ovarian disorders such as POI and DOR.

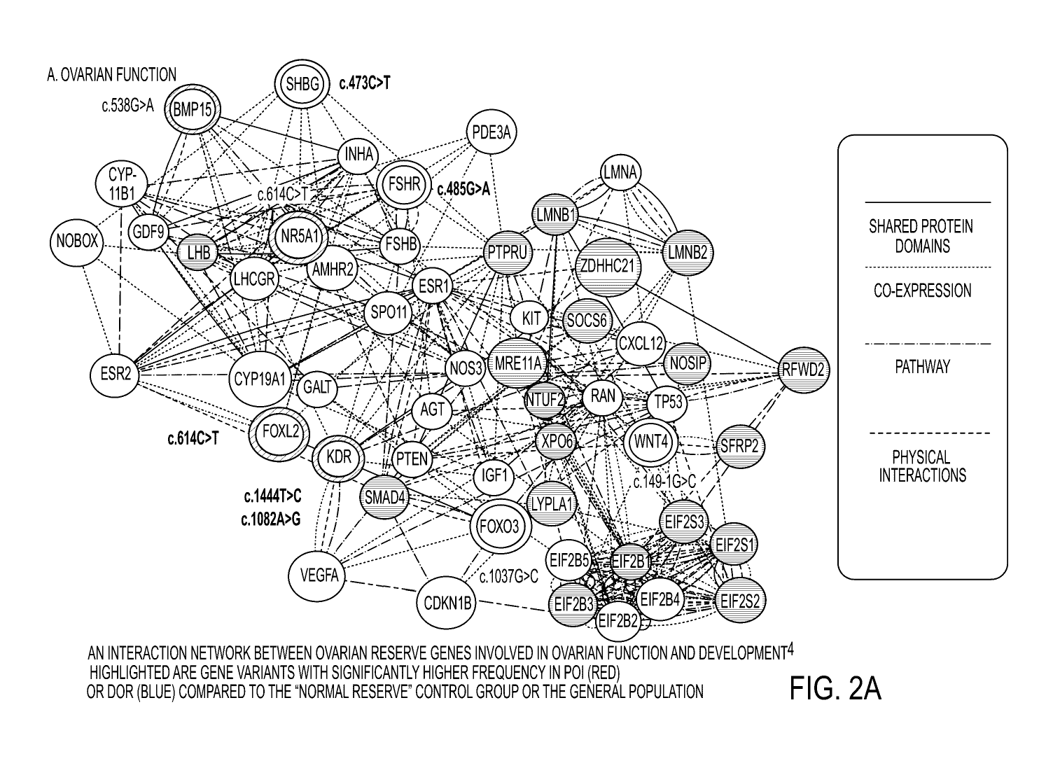

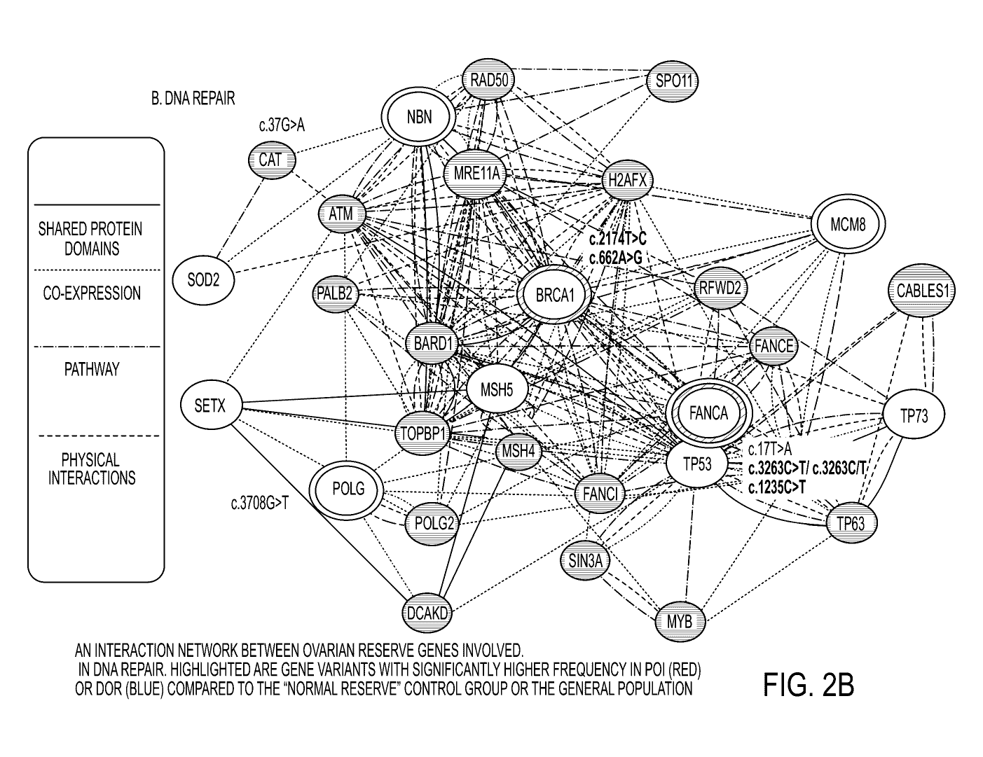

In some embodiments, biomarkers include ovarian reserve genes. Ovarian reserve genes can be any gene that affects the reserve of ovaries in a female, many of which are involved in one or more of the above described processes. Exemplary genes include, but are not limited to, BMP15, FSHR, SHBG, FOXL2, KDR, NR5A1, WNT4, FOXO3, NBN, BRCA1, FANCA, MCM8, POLG, and others, as shown in FIGS. 2A and 2B.

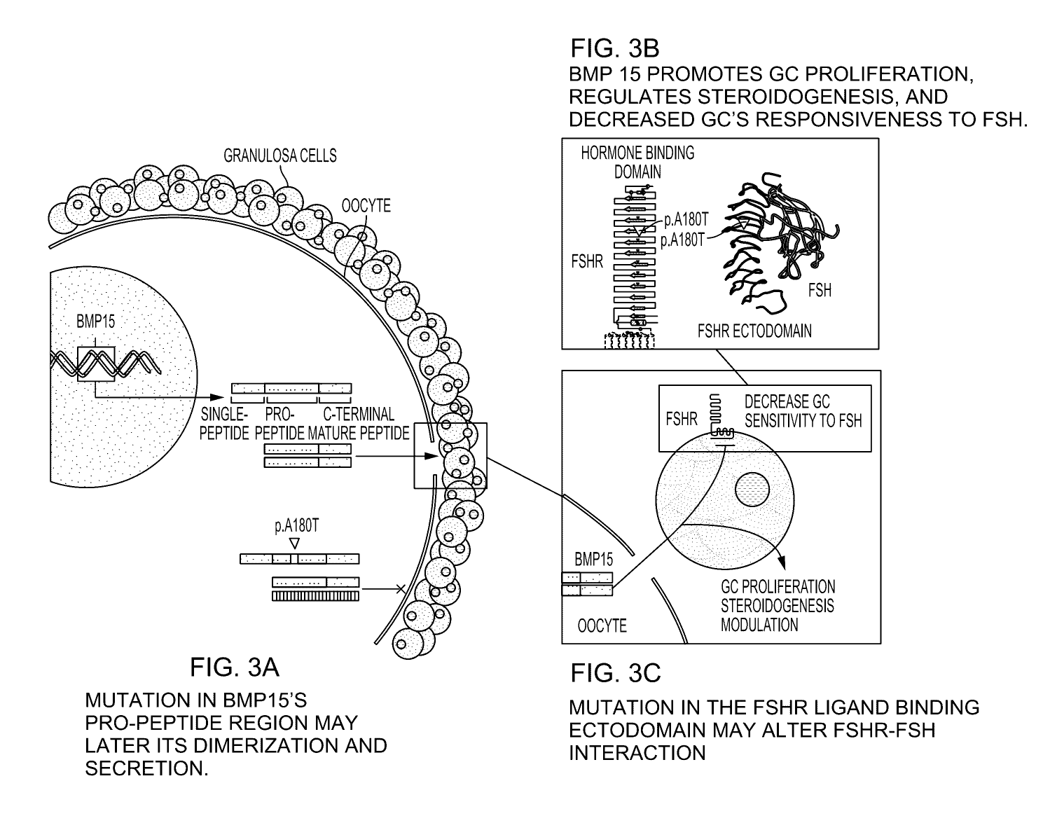

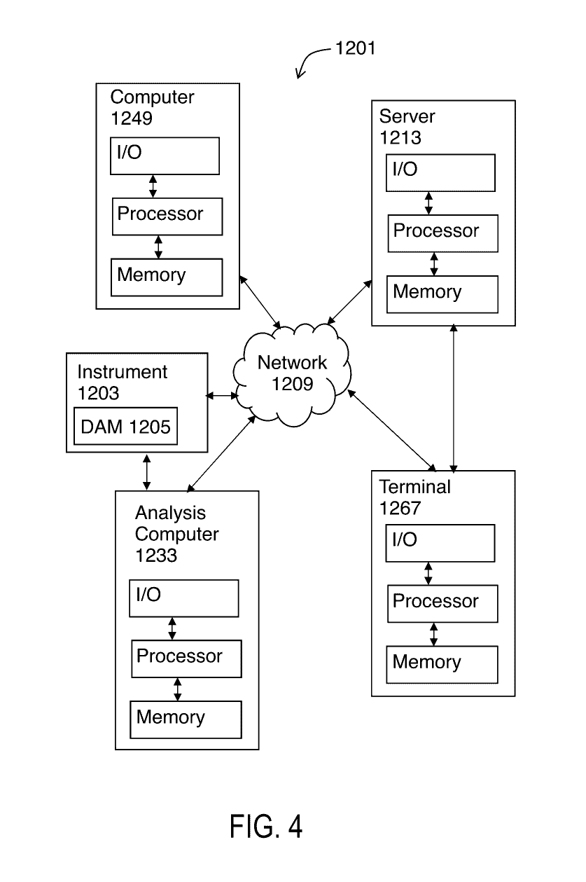

In one embodiment, biomarkers include the bone morphogenetic protein (BMP15) gene and the follicle stimulating hormone receptor (FSHR) gene. As shown in FIG. 3A, BMP15 is produced by the oocyte and is secreted into the follicular fluid. In addition to promoting granulose cell (GC) proliferation, BMP15 downregulates FSHR expression within these cells by decreasing their responsiveness to FSH, as shown in FIG. 3B. In accordance with aspects of the invention, in patients diagnosed with POI, the BMP15 mutation leads to an amino acid change from alanine to threonine at position 180 in the pro-region of BMP15, as shown in FIG. 3A. This mutation was previously associated with POI and was believed to alter BMP15 dimerization and secretion and hence its ability to act on GCs. Also in accordance with aspects of the invention, in patients diagnosed with DOR, the FSHR mutation detected in the DOR group leads to an amino acid change at position 162 from arginine to a lysine in the FSH binding region of the FSHR ectodomain, as shown in FIG. 3B. It is believed that this mutation alters the FSHR-FSH interaction.

Biomarkers according to the invention also include inflammatory genes (e.g., genes associated with inflammation processes). Exemplary inflammatory genes include, but are not limited to those genes in the interleukin 1 family such as interleukin 1 alpha (IL-1A) and interleukin-18 (IL-18); intercellular adhesion molecule 1 (ICAM1); and those in the tumor necrosis (TNF) family. Biomarkers according to the invention also include those genes of the transforming growth-factor family, such as growth differentiation factor-9 (GDF9) and genes of the inhibin family, such as inhibin, alpha (INHA).

### Obtaining Genetic Data

Genetic data can be obtained, for example, by conducting an assay on a sample from a male or female that detects either a variant in an infertility-associated/ovarian reserve genetic region or abnormal (over or under) expression of an infertility-associated/ovarian reserve genetic region. The presence of certain variants in those genetic regions or abnormal expression levels of those genetic regions is indicative of infertility/a decline in ovarian reserve and function. Exemplary variants include, but are not limited to, a single nucleotide polymorphism, a single nucleotide variant, a deletion, an insertion, an inversion, a genetic rearrangement, a copy number variation, chromosomal microdeletion, genetic mosaicism, karyotype abnormality, or a combination thereof.

In certain embodiments, a variant in a single genetic region disclosed above in the genetic data section indicates infertility/a decline in ovarian reserve and function. In other embodiments, the assay is conducted on more than one genetic region disclosed above in the genetic data section (e.g., 2, 5, 10, 20, 30, 40, 50, 60, 70, 80, all of the genes in the genetic data section, including the genes in Table 1), and the presence of variants in at least two of the genetic regions disclosed above in the genetic data section indicates infertility/a premature decline in ovarian reserve and function. In other embodiments, the presence of variants in at least three of the genetic regions disclosed above in the genetic data section indicates infertility/a premature decline in ovarian reserve and function; the presence of variants in at least four of the genetic regions disclosed above in the genetic data section indicates infertility/a premature decline in ovarian reserve and function; the presence of variants in at least five of the genetic regions disclosed above in the genetic data section indicates infertility/a premature decline in ovarian reserve and function; the presence of variants in at least six of the genetic regions disclosed above in the genetic data section indicates infertility/a premature decline in ovarian reserve and function; the presence of variants in at least seven of the genetic regions disclosed above in the genetic data section indicates infertility/a premature decline in ovarian reserve and function; the presence of variants in at least eight of the genetic regions disclosed above in the genetic data section indicates infertility/a premature decline in ovarian reserve and function; the presence of variants in at least nine of the genetic regions disclosed above in the genetic data section indicates infertility/a premature decline in ovarian reserve and function; the presence of variants in at least 10 of the genetic regions disclosed above in the genetic data section indicates infertility/a premature decline in ovarian reserve and function; the presence of variants in at least 15 of the genetic regions disclosed above in the genetic data section indicates infertility/a premature decline in ovarian reserve and function; or the presence of variants in all of the genetic regions disclosed above in the genetic data section indicates infertility/a premature decline in ovarian reserve and function.

A sample may include a human tissue or bodily fluid and may be collected in any clinically acceptable manner. A tissue is a mass of connected cells and/or extracellular matrix material, e.g. skin tissue, hair, nails, nasal passage tissue, CNS tissue, neural tissue, eye tissue, liver tissue, kidney tissue, placental tissue, mammary gland tissue, placental tissue, mammary gland tissue, gastrointestinal tissue, musculoskeletal tissue, genitourinary tissue, bone marrow, and the like, derived from, for example, a human or other mammal and includes the connecting material and the liquid material in association with the cells and/or tissues. A body fluid is a liquid material derived from, for example, a human or other mammal. Such body fluids include, but are not limited to, mucous, blood, plasma, serum, serum derivatives, bile, blood, maternal blood, phlegm, saliva, sputum, sweat, amniotic fluid, menstrual fluid, mammary fluid, follicular fluid of the ovary, fallopian tube fluid, peritoneal fluid, urine, semen, and cerebrospinal fluid (CSF), such as lumbar or ventricular CSF. A sample may also be a fine needle aspirate or biopsied tissue, e.g. an endometrial aspirate, breast tissue biopsy, and the like. A sample also may be media containing cells or biological material. A sample may also be a blood clot, for example, a blood clot that has been obtained from whole blood after the serum has been removed. In certain embodiments, the sample may include reproductive cells or tissues, such as gametic cells, gonadal tissue, fertilized embryos, and placenta. In certain embodiments, the sample is blood, saliva, or semen collected from the subject.

Genetic information from the sample can be obtained by nucleic acid extraction from the sample. Methods for extracting nucleic acid from a sample are known in the art. See for example, Maniatis, et al., Molecular Cloning: A Laboratory Manual, Cold Spring Harbor, N.Y., pp. 280-281, 1982, the contents of which are incorporated by reference herein in their entirety. In certain embodiments, a sample is collected from a subject followed by enrichment for genes or gene fragments of interest, for example by hybridization to a nucleotide array including fertility-related genetic regions or genetic fragments of interest. The sample may be enriched for genetic regions of interest (e.g., infertility-associated genetic regions) using methods known in the art, such as hybrid capture. See for examples, Lapidus (U.S. Pat. No. 7,666,593), the content of which is incorporated by reference herein in its entirety.

In particular embodiments, the assay is conducted on genes or genetic regions containing the gene or a part thereof associated with infertility and/or ovarian function or reserve, such as those genes found in Tables 1 and/or specifically enumerated. Detailed descriptions of conventional methods, such as those employed to make and use nucleic acid arrays, amplification primers, hybridization probes, and the like can be found in standard laboratory manuals such as: Genome Analysis: A Laboratory Manual Series (Vols. I-IV), Cold Spring Harbor Laboratory Press; PCR Primer: A Laboratory Manual, Cold Spring Harbor Laboratory Press; and Sambrook, J et al., (2001) Molecular Cloning: A Laboratory Manual, 2nd ed. (Vols. 1-3), Cold Spring Harbor Laboratory Press. Custom nucleic acid arrays are commercially available from, e.g., Affymetrix (Santa Clara, Calif.), Applied Biosystems (Foster City, Calif.), and Agilent Technologies (Santa Clara, Calif.).

Methods of detecting variations (e.g., mutations) are known in the art. In certain embodiments, a known single nucleotide polymorphism at a particular position can be detected by single base extension for a primer that binds to the sample DNA adjacent to that position. See for example Shuber et al. (U.S. Pat. No. 6,566,101), the content of which is incorporated by reference herein in its entirety. In other embodiments, a hybridization probe might be employed that overlaps the SNP of interest and selectively hybridizes to sample nucleic acids containing a particular nucleotide at that position. See for example Shuber et al. (U.S. Pat. Nos. 6,214,558 and 6,300,077), the content of which is incorporated by reference herein in its entirety.

In particular embodiments, nucleic acids are sequenced in order to detect variants in the nucleic acid compared to wild-type and/or non-mutated forms of the sequence. The nucleic acid can include a plurality of nucleic acids derived from a plurality of genetic elements. Methods of detecting sequence variants are known in the art, and sequence variants can be detected by any sequencing method known in the art.

DNA sequencing techniques include classic dideoxy sequencing reactions (Sanger method) using labeled terminators or primers and gel separation in slab or capillary, sequencing by synthesis using reversibly terminated labeled nucleotides, pyrosequencing, allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLiD sequencing. Sequencing of separated molecules has more recently been demonstrated by sequential or single extension reactions using polymerases or ligases as well as by single or sequential differential hybridizations with libraries of probes

One conventional method to perform sequencing is by chain termination and gel separation, as described by Sanger et al., Proc Natl. Acad. Sci. USA, 74(12): 5463 67 (1977). Another conventional sequencing method involves chemical degradation of nucleic acid fragments. See, Maxam et al., Proc. Natl. Acad. Sci., 74: 560 564 (1977). Finally, methods have been developed based upon sequencing by hybridization. See, e.g., Harris et al., (U.S. patent application number 2009/0156412). The content of each reference is incorporated by reference herein in its entirety.

A sequencing technique that can be used in the methods of the provided invention includes, for example, Helicos True Single Molecule Sequencing (tSMS) (Harris T. D. et al. (2008) Science 320:106-109), incorporated herein by reference; see also, e.g., Lapidus et al. (U.S. Pat. No. 7,169,560), Lapidus et al. (U.S. patent application number 2009/0191565), Quake et al. (U.S. Pat. No. 6,818,395), Harris (U.S. Pat. No. 7,282,337), Quake et al. (U.S. patent application number 2002/0164629), and Braslaysky, et al., PNAS (USA), 100: 3960-3964 (2003), the contents of each of these references is incorporated by reference herein in its entirety. Another example of a DNA sequencing technique that can be used in the methods of the provided invention is 454 sequencing (Roche) (Margulies, M et al. 2005, Nature, 437, 376-380).

Another example of a DNA sequencing technique that can be used in the methods of the provided invention is SOLiD technology (Applied Biosystems). Another example of a DNA sequencing technique that can be used in the methods of the provided invention is Ion Torrent sequencing (U.S. patent application numbers 2009/0026082, 2009/0127589, 2010/0035252, 2010/0137143, 2010/0188073, 2010/0197507, 2010/0282617, 2010/0300559), 2010/0300895, 2010/0301398, and 2010/0304982), the content of each of which is incorporated by reference herein in its entirety.

Another example of a sequencing technology that can be used in the methods of the provided invention is next-generation sequencing (NGS), such as Illumina sequencing, using Illumina HiSeq sequencers. Illumina sequencing is based on the amplification of DNA on a solid surface using fold-back PCR and anchored primers. Genomic DNA is fragmented, and adapters are added to the 5′ and 3′ ends of the fragments. DNA fragments that are attached to the surface of flow cell channels are extended and bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured and the identity of the first base is recorded. The 3′ terminators and fluorophores from each incorporated base are removed and the incorporation, detection and identification steps are repeated.

Another example of a sequencing technology that can be used in the methods of the provided invention includes the single molecule, real-time (SMRT) technology of Pacific Biosciences. In SMRT, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in an out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

Another example of a sequencing technique that can be used in the methods of the provided invention is nanopore sequencing (Soni G V and Meller A. (2007) Clin Chem 53: 1996-2001, incorporated herein by reference). Another example of a sequencing technique that can be used in the methods of the provided invention involves using a chemical-sensitive field effect transistor (chemFET) array to sequence DNA (for example, as described in US Patent Application Publication No. 20090026082 and incorporated by reference). Another example of a sequencing technique that can be used in the methods of the provided invention involves using an electron microscope (Moudrianakis E. N. and Beer M. Proc Natl Acad Sci USA. 1965 March; 53:564-71, incorporated herein by reference).

In certain aspects, the invention provides a microarray including a plurality of oligonucleotides attached to a substrate at discrete addressable positions, in which at least one of the oligonucleotides hybridizes to a portion of a gene suspected of affecting fertility in a man or woman. Methods of constructing microarrays are known in the art. See for example Yeatman et al. (U.S. patent application number 2006/0195269), the content of which is hereby incorporated by reference in its entirety.

If the nucleic acid from the sample is degraded or only a minimal amount of nucleic acid can be obtained from the sample, PCR can be performed on the nucleic acid in order to obtain a sufficient amount of nucleic acid for sequencing (See e.g., Mullis et al. U.S. Pat. No. 4,683,195, the contents of which are incorporated by reference herein in its entirety).

Sequencing by any of the methods described above and known in the art produces sequence reads. Sequence reads can be analyzed to call variants by any number of methods known in the art. Variant calling can include aligning sequence reads to a reference (e.g. hg18) and reporting variants, such as single nucleotide polymorphism (SNP)/single nucleotide variant (SNV) alleles. An example of methods for analyzing sequence reads and calling variants includes standard Genome Analysis Toolkit (GATK) methods. See The Genome Analysis Toolkit: a MapReduce framework for analyzing next-generation DNA sequencing data, Genome Res 20(9):1297-1303, the contents of each of which are incorporated by reference. GATK is a software package for analysis of high-throughput sequencing data capable of identifying variants, including SNPs.

Variants can be reported in a format such as a Sequence Alignment Map (SAM) or a Variant Call Format (VCF) file. Some background may be found in Li & Durbin, 2009, Fast and accurate short read alignment with Burrows-Wheeler Transform. Bioinformatics 25:1754-60 and McKenna et al., 2010. Variant calling produces results (“variant calls”) that may be stored as a sequence alignment map (SAM) or binary alignment map (BAM) file—comprising an alignment string (the SAM format is described, e.g., in Li, et al., The Sequence Alignment/Map format and SAMtools, Bioinformatics, 2009, 25(16):2078-9). Additionally or alternatively, output from the variant calling may be provided in a variant call format (VCF) file, e.g., in report. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described in Danecek et al., 2011, The variant call format and VCFtools, Bioinformatics 27(15):2156-2158. Further discussion may be found in U.S. Pub. 2013/0073214; U.S. Pub. 2013/0345066; U.S. Pub. 2013/0311106; U.S. Pub. 2013/0059740; U.S. Pub. 2012/0157322; U.S. Pub. 2015/0057946 and U.S. Pub. 2015/0056613, each incorporated by reference.

Once the variants, such as SNPs/SNVs, have been identified, deleterious variants can be determined by any number of methods known in the art. One example of a method for determining deleterious SNPs/SNVs is through the use of SnpEff, a genetic variant annotation and effect prediction toolbox. SnpEff is capable of rapidly categorizing the effects of SNPs/SNVs and other variants in whole genome sequences. See, Cingolani et al., A program for annotating and predicting the effects of single nucleotide polymorphisms, SnpEff: SNPs in the genome of Drosophila melanogaster strain w¹¹¹⁸; iso-2; iso-3; Landes Bioscience, 6:2, 1-13; April/May/June 2012, incorporated herein by reference.

Upon identification of deleterious variants such as SNPs/SNVs, the variants can be filtered for those that are fertility-centric. One of ordinary skill in the art would understand that both molecular and computational approaches are available for filtering variants. One of ordinary skill in the art would also understand how to filter deleterious variants for fertility centric genes (e.g. by comparing to a known database, through the use of ANOVA technology, through the use of multivariate analysis). It is to be understood that various fertility-centric bioinformatics pipelines incorporating pathway analysis tools can be used to filter deleterious variants in accordance with the invention. See e.g., U.S. patent application Ser. Nos. 14/107,800, 14/802,609, 15/209,357, 62/381,916, and 62/408,632, all of which are incorporated herein in their entirety.

Furthermore, in one aspect of the invention, genes of interest can be annotated into functional pathways using any method known in the art. One example of a pathway analysis tool for gene annotation includes the Database for Annotation, Visualization and Integrated Discover (DAVID). Nature Protocols 2009; 4(1):44; and Nucleic Acids Res. 2009; 37(1):1, incorporated herein by references.

Methods of the invention also include conducting an assay on a sample from a subject that detects an abnormal (over or under) expression of an infertility-associated gene (e.g. a differentially or abnormally expressed gene). A differentially or abnormally expressed gene refers to a gene whose expression is activated to a higher or lower level in a subject suffering from a disorder, such as infertility, relative to its expression in a normal or control subject. The terms also include genes whose expression is activated to a higher or lower level at different stages of the same disorder. It is also understood that a differentially expressed gene may be either activated or inhibited at the nucleic acid level or protein level, or may be subject to alternative splicing to result in a different polypeptide product. Such differences may be evidenced by a change in mRNA levels, surface expression, secretion or other partitioning of a polypeptide, for example.

Differential gene expression may include a comparison of expression between two or more genes or their gene products, or a comparison of the ratios of the expression between two or more genes or their gene products, or even a comparison of two differently processed products of the same gene, which differ between normal subjects and subjects suffering from a disorder, such as infertility, or between various stages of the same disorder. Differential expression includes both quantitative, as well as qualitative, differences in the temporal or cellular expression pattern in a gene or its expression products. Differential gene expression (increases and decreases in expression) is based upon percent or fold changes over expression in normal cells. Increases may be of 1, 5, 10, 20, 30, 40, 50, 60, 70, 80, 90, 100, 120, 140, 160, 180, or 200% relative to expression levels in normal cells. Alternatively, fold increases may be of 1, 1.5, 2, 2.5, 3, 3.5, 4, 4.5, 5, 5.5, 6, 6.5, 7, 7.5, 8, 8.5, 9, 9.5, or 10 fold over expression levels in normal cells. Decreases may be of 1, 5, 10, 20, 30, 40, 50, 55, 60, 65, 70, 75, 80, 82, 84, 86, 88, 90, 92, 94, 96, 98, 99 or 100% relative to expression levels in normal cells.

Methods of detecting levels of gene products (e.g., RNA or protein) are known in the art. Commonly used methods known in the art for the quantification of mRNA expression in a sample include northern blotting and in situ hybridization (Parker & Barnes, Methods in Molecular Biology 106:247 283 (1999), the contents of which are incorporated by reference herein in their entirety); RNAse protection assays (Hod, Biotechniques 13:852 854 (1992), the contents of which are incorporated by reference herein in their entirety); and PCR-based methods, such as reverse transcription polymerase chain reaction (RT-PCR) (Weis et al., Trends in Genetics 8:263 264 (1992), the contents of which are incorporated by reference herein in their entirety). Alternatively, antibodies may be employed that can recognize specific duplexes, including RNA duplexes, DNA-RNA hybrid duplexes, or DNA-protein duplexes. Other methods known in the art for measuring gene expression (e.g., RNA or protein amounts) are shown in Yeatman et al. (U.S. patent application number 2006/0195269), the content of which is hereby incorporated by reference in its entirety.

In certain embodiments, reverse transcriptase PCR (RT-PCR) is used to measure gene expression. RT-PCR is a quantitative method that can be used to compare mRNA levels in different sample populations to characterize patterns of gene expression, to discriminate between closely related mRNAs, and to analyze RNA structure. Various methods are well known in the art. See, e.g., Ausubel et al., Current Protocols of Molecular Biology, John Wiley and Sons (1997); Rupp and Locker, Lab Invest. 56:A67 (1987), and De Andres et al., BioTechniques 18:42044 (1995); Held et al., Genome Research 6:986 994 (1996), the contents of which are incorporated by reference herein in their entirety.

Further PCR-based techniques include, for example, differential display (Liang and Pardee, Science 257:967 971 (1992)); amplified fragment length polymorphism (iAFLP) (Kawamoto et al., Genome Res. 12:1305 1312 (1999)); BeadArray™ technology (Illumina, San Diego, Calif.; Oliphant et al., Discovery of Markers for Disease (Supplement to Biotechniques), June 2002; Ferguson et al., Analytical Chemistry 72:5618 (2000)); BeadsArray for Detection of Gene Expression (BADGE), using the commercially available Luminex100 LabMAP system and multiple color-coded microspheres (Luminex Corp., Austin, Tex.) in a rapid assay for gene expression (Yang et al., Genome Res. 11:1888 1898 (2001)); and high coverage expression profiling (HiCEP) analysis (Fukumura et al., Nucl. Acids. Res. 31(16) e94 (2003)). The contents of each of which are incorporated by reference herein in their entirety.

In another embodiment, a MassARRAY-based gene expression profiling method is used to measure gene expression. For further details see, e.g. Ding and Cantor, Proc. Natl. Acad. Sci. USA 100:3059 3064 (2003), incorporated herein by reference.

In certain embodiments, differential gene expression can also be identified, or confirmed using a microarray technique. In this method, polynucleotide sequences of interest (including cDNAs and oligonucleotides) are plated, or arrayed, on a microchip substrate. The arrayed sequences are then hybridized with specific DNA probes from cells or tissues of interest. Methods for making microarrays and determining gene product expression (e.g., RNA or protein) are shown in Yeatman et al. (U.S. patent application number 2006/0195269), the content of which is incorporated by reference herein in its entirety. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2):106 149 (1996), the contents of which are incorporated by reference herein in their entirety). Microarray analysis can be performed by commercially available equipment, following manufacturer's protocols, such as by using the Affymetrix GenChip technology, or Incyte's microarray technology.

In another aspect, protein levels can be determined by constructing an antibody microarray in which binding sites comprise immobilized, preferably monoclonal, antibodies specific to a plurality of protein species encoded by the cell genome. Preferably, antibodies are present for a substantial fraction of the proteins of interest. Methods for making monoclonal antibodies are well known (see, e.g., Harlow and Lane, 1988, ANTIBODIES: A LABORATORY MANUAL, Cold Spring Harbor, N.Y., which is incorporated in its entirety for all purposes).

In yet another aspect, levels of transcripts of marker genes in a number of tissue specimens may be characterized using a “tissue array” (Kononen et al., Nat. Med 4(7):844-7 (1998)). In a tissue array, multiple tissue samples are assessed on the same microarray. The arrays allow in situ detection of RNA and protein levels; consecutive sections allow the analysis of multiple samples simultaneously.

In other embodiments, Serial Analysis of Gene Expression (SAGE) is used to measure gene expression. Serial analysis of gene expression (SAGE) is a method that allows the simultaneous and quantitative analysis of a large number of gene transcripts, without the need of providing an individual hybridization probe for each transcript. For more details see, e.g. Velculescu et al., Science 270:484 487 (1995); and Velculescu et al., Cell 88:243 51 (1997, the contents of each of which are incorporated by reference herein in their entirety).

In other embodiments, Massively Parallel Signature Sequencing (MPSS) is used to measure gene expression. For more details see, e.g. Brenner et al., Nature Biotechnology 18:630 634 (2000).

Immunohistochemistry methods are also suitable for detecting the expression levels of the gene products of the present invention. In these methods, antibodies (monoclonal or polyclonal) or antisera, such as polyclonal antisera, specific for each marker are used to detect expression. Immunohistochemistry protocols and kits are well known in the art and are commercially available.

In certain embodiments, a proteomics approach is used to measure gene expression. A proteome refers to the totality of the proteins present in a sample (e.g. tissue, organism, or cell culture) at a certain point of time. Proteomics includes, among other things, study of the global changes of protein expression in a sample (also referred to as expression proteomics). Proteomics typically includes the following steps: (1) separation of individual proteins in a sample by 2-D gel electrophoresis (2-D PAGE); (2) identification of the individual proteins recovered from the gel, e.g. my mass spectrometry or N-terminal sequencing, and (3) analysis of the data using bioinformatics. Proteomics methods are valuable supplements to other methods of gene expression profiling, and can be used, alone or in combination with other methods, to detect the products of the prognostic markers of the present invention.

In some embodiments, mass spectrometry (MS) analysis can be used alone or in combination with other methods (e.g., immunoassays or RNA measuring assays) to determine the presence and/or quantity of the one or more biomarkers disclosed herein in a biological sample. In some embodiments, the MS analysis includes matrix-assisted laser desorption/ionization (MALDI) time-of-flight (TOF) MS analysis, such as for example direct-spot MALDI-TOF or liquid chromatography MALDI-TOF mass spectrometry analysis. In some embodiments, the MS analysis comprises electrospray ionization (ESI) MS, such as for example liquid chromatography (LC) ESI-MS. Mass analysis can be accomplished using commercially-available spectrometers. Methods for utilizing MS analysis, including MALDI-TOF MS and ESI-MS, to detect the presence and quantity of biomarker peptides in biological samples are known in the art. See, for example, U.S. Pat. Nos. 6,925,389; 6,989,100; and 6,890,763, each of which is incorporated by reference herein in their entirety.

### Clinical Information

Assessment and analysis of likelihood of achieving ongoing pregnancy and live birth also incorporates the use of clinical fertility-associated information, such as phenotypic and/or environmental characteristics. Exemplary clinical information is provided in Table 2 below.

TABLE 2
Clinical information

Cholesterol levels on different days of the menstrual cycle
Age of first menses for patient and female blood relatives (e.g. sisters, mother, grandmothers)
Age of menopause for female blood relatives (e.g. sisters, mother, grandmothers)
Number of previous pregnancies (biochemical/ectopic/clinical/fetal heart beat detected, live
birth outcomes), age at the time, and outcome for patient and female blood relatives (e.g. sisters,
mother, grandmothers)
Diagnosis of Polycystic Ovarian Syndrome
bAFC
number of embryos transferred
PGS
Female Hormone levels, such as AMH, LSH, FSH and E2
History of hydrosalpinx or tubal occlusion
History of endometriosis, pelvic pain, or painful periods
Cancer history/type of cancer/treatment/outcome for patient and female blood relatives (e.g.
sisters, mother, grandmothers)
Age that sexual activity began, current level of sexual activity
Smoking history for patient and blood relatives
Travel schedule/number of flying hours a year/time difference changes of more than 3 hours
(Jetlag and Flight-associated Radiation Exposure)
Nature of periods (length of menses, length of cycle)
Biological age (number of years since first menses)
Birth control use
Drug use (illegal or legal)
Body mass index (current, lowest ever, highest ever)
History of polyps
History of hormonal imbalance
History of amenorrhoea
History of eating disorders
Alcohol consumption by patient or blood relatives
Details of mother's pregnancy with patient (i.e. measures of uterine environment): any drugs
taken, smoking, alcohol, stress levels, exposure to plastics (i.e. Tupperware), composition of
diet (see below)
Sleep patterns: number of hours a night, continuous/overall
Diet: meat, organic produce, vegetables, vitamin or other supplement consumption, dairy (full
fat or reduced fat), coffee/tea consumption, folic acid, sugar (complex, artificial, simple),
processed food versus home cooked.
Exposure to plastics: microwave in plastic, cook with plastic, store food in plastic, plastic water
or coffee mugs.
Water consumption: amount per day, format: straight from the tap, bottled water (plastic or
bottle), filtered (type: e.g. Britta/Pur)
Residence history starting with mother's pregnancy: location/duration
Environmental exposure to potential toxins for different regions (extracted from government
monitoring databases)
Health metrics: autoimmune disease, chronic illness/condition
Pelvic surgery history
Life time number of pelvic X-rays
History of sexually transmitted infections: type/treatment/outcome
Female reproductive hormone levels: follicle stimulating hormone, anti-Mullerian hormone,
estrogen, progesterone
Stress
Thickness and type of endometrium throughout the menstrual cycle.
Age
Height
Fertility treatment history and details: history of hormone stimulation, brand of drugs used,
basal antral follicle count, follicle count after stimulation with different protocols,
number/quality/stage of retrieved oocytes/development profile of embryos resulting from in
vitro insemination (natural or ICSI), details of IVF procedure (which clinic,
doctor/embryologist at clinic, assisted hatching, fresh or thawed oocytes/embryos, embryo
transfer (blood on the catheter/squirt detection and direction on ultrasound), number of
successful and unsuccessful IVF attempts
Morning sickness during pregnancy
Breast size before/during/after pregnancy
History of ovarian cysts
Twin or sibling from multiple birth (mono-zygotic or di-zygotic)
Semen analysis (count, motility, morphology)
Vasectomy
Testosterone levels
Date of last use and/or frequency of use of a hot tub or sauna
Blood type
DES exposure in utero
Past and current exercise/athletic history
Levels of phthalates, including metabolites:
MEP - monoethyl phthalate, MECPP - mono(2-ethyl-5-carboxypentyl) phthalate, MEHHP -
mono(2-ethyl-5-hydroxyhexyl) phthalate, MEOHP - mono(2-ethyl-5-ox-ohexyl) phthalate,
MBP - monobutyl phthalate, MBzP - monobenzyl phthalate, MEHP - mono(2-ethylhexyl)
phthalate, MiBP - mono-isobutyl phthalate, MCPP - mono(3-carboxypropyl) phthalate, MCOP -
monocarboxyisooctyl phthalate, MCNP - monocarboxyisononyl phthalate
Familial history of Premature Ovarian Failure/Insufficiency
Autoimmunity history - Antiadrenal antibodies (anti-21-hydroxylase antibodies), antiovarian
antibodies, antithyroid anitibodies (anti-thyroid peroxidase, antithyroglobulin)
Additional female hormone levels: Leutenizing hormone (using immunofluorometric assay),
Δ4-Androstenedione (using radioimmunoassay), Dehydroepiandrosterone (using
radioimmunoassay), and Inhibin B (commercial ELISA)
Number of years trying to conceive
Dioxin and PVC exposure
Hair color
Nevi (moles)
Lead, cadmium, and other heavy metal exposure
For a particular ART cycle: the percentage of eggs that were abnormally fertilized, if assisted
hatching was performed, if anesthesia was used, average number of cells contained by the
embryo at the time of cryopreservation, average degree of expansion for blastocyst represented
as a score, average degree of expansion of a previously frozen embryo represented as a score,
embryo quality metrics including but not limited to degree of cell fragmentation and
visualization of a or organization/number of cells contained in the inner cell mass (ICM), the
fraction of overall embryos that make it to the blastocyst stage of development, the number of
embryos that make it to the blastocyst stage of development, use of birth control, the brand
name of the hormones used in ovulation induction, hyperstimulation syndrome, reason for
cancelation of a treatment cycle, chemical pregnancy detected, clinical pregnancy detected,
count of germinal vesicle containing oocytes upon retrieval, count of metaphase I stage eggs
upon retrieval, count of metaphase II stage eggs upon retrieval, count of embryos or oocytes
arrested in development and the stage of development or day of development post oocyte
retrieval, number of embryos transferred and date in days post-oocyte retrieval that the embryos
were transferred, how many embryos were cryopreserved and at what stage of development

Information regarding the clinical information, such as the information listed in Table 2, can be obtained by any means known in the art. In many cases, such information can be obtained from a questionnaire completed by the subject that contains questions regarding certain clinical data. Additional information can be obtained from a questionnaire completed by the subject's partner and blood relatives. The questionnaire includes questions regarding the subject's clinical traits, such as his or her age, smoking habits, or frequency of alcohol consumption. Information can also be obtained from the medical history of the subject, as well as the medical history of blood relatives and other family members. Additional information can be obtained from the medical history and family medical history of the subject's partner. Medical history information can be obtained through analysis of electronic medical records, paper medical records, a series of questions about medical history included in the questionnaire, and a combination thereof.

In other embodiments, an assay specific to a phenotypic trait or an environmental exposure of interest is used. Such assays are known to those of skill in the art, and may be used with methods of the invention. For example, the hormones may be detected from a urine or blood test. Venners et al. (Hum. Reprod. 21(9): 2272-2280, 2006) reports assays for detecting estrogen and progesterone in urine and blood samples. Venner also reports assays for detecting the chemicals used in fertility treatments. Hormones can also be detected in a saliva sample. See, for example, Yu-cai et al. (Fertility and Sterility, 71(5): 863-868, 1999); and Worthman et al., (Clin. Chem. 36(10): 1769-1773, 1990), both of which disclose methods for detecting salivary hormone levels and are incorporated herein in their entirety.

Similarly, illicit drug use may be detected from a tissue or body fluid, such as hair, urine, sweat, or blood, and there are numerous commercially available assays (LabCorp) for conducting such tests. Standard drug tests look for ten different classes of drugs, and the test is commercially known as a “10-panel urine screen”. The 10-panel urine screen consists of the following: 1. Amphetamines (including Methamphetamine) 2. Barbiturates 3. Benzodiazepines 4. Cannabinoids (THC) 5. Cocaine 6. Methadone 7. Methaqualone 8. Opiates (Codeine, Morphine, Heroin, Oxycodone, Vicodin, etc.) 9. Phencyclidine (PCP) 10. Propoxyphene. Use of alcohol can also be detected by such tests.

Numerous assays can be used to tests a patient's exposure to plastics (e.g., Bisphenol A (BPA)). BPA is most commonly found as a component of polycarbonates (about 74% of total BPA produced) and in the production of epoxy resins (about 20%). As well as being found in a myriad of products including plastic food and beverage contains (including baby and water bottles), BPA is also commonly found in various household appliances, electronics, sports safety equipment, adhesives, cash register receipts, medical devices, eyeglass lenses, water supply pipes, and many other products. Assays for testing blood, sweat, or urine for presence of BPA are described, for example, in Genuis et al. (Journal of Environmental and Public Health, Volume 2012, Article ID 185731, 10 pages, 2012).

### Assessment of Ovarian Reserve and Function—Ovarian Reserve Predictor

The genetic and clinical data collected from the female subject is then compared to a reference set of data in order to provide a probability of premature decline in ovarian reserve or function, including a probability of being diagnosed with a disorder affecting ovarian reserve or function, such as DOR or POI. In certain aspects, the reference set includes data collected from a cohort or plurality of women, some of which have been diagnosed with DOR and/or POI. Such data may include genetic data from the women, clinical information from the women, such as their age and hormone levels, and other traits listed in Table 2, fertility-associated medical interventions, their pregnancy outcome, i.e., whether or not a pregnancy or live-birth was achieved, per cycle of the selected reproductive method, and any diagnosis of an ovarian reserve or function disorder. As disclosed above, genetic and clinical information can be obtained by any means known in the art. In certain embodiments, the information is obtained via a questionnaire. In other embodiments, information can be obtained by analyzing a sample collected from the women in the reference set. In further embodiments of the invention, when data comprising the fertility-associated phenotypic traits of a male subject is obtained, the reference set will include data regarding those traits collected from a plurality of men. Additional details for preparing a mass data set for use, for example, in IVF studies are provided in Malizia et al., Cumulative live-birth rates after in vitro fertilization, N Engl J Med 2009; 360: 236-43, incorporated by reference herein in its entirety.

The invention provides methods and systems for predicting a pregnancy outcome in a female subject based on the subject's fertility and/or ovarian reserve-related clinical information and genotypic data. In some embodiments, methods and systems of the invention use an ovarian reserve predictor for predicting the probability of a subject having a disorder associated with a premature decrease in ovarian reserve or function, and ultimately infertility. The ovarian reserve predictor can be based on any appropriate pattern recognition method that receives input data representative of a plurality of clinical and genetic traits, and provides an output that indicates a probability of a subject having a disorder associated with a premature decrease in ovarian reserve or function, and ultimately infertility. The ovarian reserve predictor is trained with training data from a plurality of women for whom fertility/ovarian reserve-associated clinical information and/or genetic data, fertility-associated medical interventions, ovarian reserve or function disorder diagnoses, and pregnancy outcomes are known. Various ovarian reserve predictors that can be used in conjunction with the present invention are described below. In some embodiments, additional women having known profiles, diagnoses, and pregnancy outcomes can be used to test the accuracy of the ovarian reserve predictor obtained using the training population. Such additional patients are known as the testing population.

In certain embodiments, the methods of invention use the ovarian reserve predictor for determining the probability of premature decline in ovarian reserve or function and/or having a disorder that affects ovarian reserve or function, such as DOR or POI. As noted above, the ovarian reserve predictor can be based on any appropriate pattern recognition method that receives a profile, such as a profile based on a plurality of fertility/ovarian reserve-associated genetic and clinical traits and provides an output comprising data indicating a good prognosis or a poor prognosis, i.e., whether or not the individual has a risk of premature decline in ovarian reserve or function or is more likely to be diagnosed with a disorder that affects ovarian reserve or function. As discussed previously, the profile can be obtained by completion of a questionnaire containing questions regarding certain fertility/ovarian reserve-associated clinical traits or the collection of a biological sample to obtain genotypic data or a combination thereof. The ovarian reserve predictor is trained with training data from a training population of women for whom fertility/ovarian-associated genetic and clinical traits, fertility-associated medical interventions, and diagnoses are known.

A prognosis predictor based on any of such methods can be constructed using the profiles and diagnoses data of the training patients. Such an ovarian reserve predictor can then be used to predict the probability of a female subject having a disorder which affects ovarian reserve or function based on her profile of fertility-associated phenotypic traits, genotypic traits, or both. The methods can also be used to identify traits that discriminate between having a disorder or not having a disorder using a trait profile and prognosis data of the training population.

In one embodiment, the ovarian reserve predictor can be prepared by (a) generating a reference set of women for whom fertility/ovarian reserve-associated clinical and/or genetic characteristics, fertility-associated medical interventions, ovarian reserve or function disorder diagnoses, and pregnancy outcomes are known; (b) determining for each characteristic or characteristics, a metric of correlation between the characteristic(s) and a diagnosis of a disorder associated with a decline in ovarian reserve or function (e.g. DOR and POI) in a plurality of women having known diagnoses; (c) selecting one or more characteristics based on said level of correlation; (d) training the ovarian reserve predictor, in which the ovarian reserve predictor receives data representative of the characteristic(s) selected in the prior step and provides an output indicating a probability of having a disorder that affects ovarian reserve or function, with training data from the reference set of subjects including assessments of characteristics taken from the women.

Various known statistical pattern recognition methods can be used in conjunction with the present invention to assess ovarian reserve and function. Exemplary statistical methods include, without limitation, generalized linear models (e.g., logistic regression, ordinal logistic regression, Poisson regression, gamma regression, ordinary least squares), least absolute shrinkage and selection operator (lasso) regression, clustering, principal component analysis, nearest neighbor classifier analysis, and classification and regression trees (CARTs). Non-limiting examples of implementing particular ovarian reserve predictors are provided herein to demonstrate the implementation of statistical methods.

In some embodiments, the ovarian reserve predictor is based on a regression model, such as a logistic regression model, which can be used to estimate the odds of a patient being diagnosed with DOR or POI, given the presence of clinical and/or genetic markers. Such a regression model includes coefficients for the genetic markers, clinical markers, and/or a combination thereof, in a selected set of markers of the invention. In such embodiments, the coefficients for the regression model are computed using, for example, a maximum likelihood approach.

Some embodiments of the present invention provide generalizations of the logistic regression model that handle multicategory (polychotomous) responses. Such embodiments can be used to discriminate an organism into one, two, three, four, or more prognosis groups. Such regression models use multicategory logit models which simultaneously refer to all pairs of categories, and describe the odds of response in one category instead of the other. Once the model specifies logits for a certain (J-1) pairs of categories, the rest are redundant. See, for example, Agresti, An Introduction to Categorical Data Analysis, John Wiley & Sons, Inc., 1996, New York, Chapter 8, which is hereby incorporated by reference.

Some embodiments of the present invention treat ovarian reserve as a continuous or a count variable in a regression model relating genetic markers to metrics known to be measures of ovarian reserve. Non-limiting examples of measures of ovarian reserve include, for example, basal antral follicle count (bAFC) and anti-Müllerian hormone (AMH) levels. Such a regression model includes coefficients for the genetic markers, clinical markers, and/or a combination thereof, in a selected set of markers of the invention for predicting measures of ovarian reserve. In such embodiments, Poisson regression may, for example, be used to assess the correlation between genetic markers and bAFC.

Some embodiments of the current invention may utilize Bayesian methods to estimate the correlation between genetic markers and an increased likelihood of DOR, POI, or measures of ovarian reserve. Bayesian methods result in estimates of the so-called posterior distribution of parameters. Posterior distributions of estimates allow analysts to make probabilistic claims as to the likely values of parameters, for example, the probability that the parameter θ exceeds some value. The posterior distribution for a parameter, θ, given observed data, X, is:

P(θ|X)=[P(X|θ)P(θ)]/P(X),

Where P(X|θ) is known as the likelihood function, P(θ) the prior distribution of θ, and P(X) a normalization constant to ensure P(θ|X) integrates to unity. Bayesian methods require for the specification of a prior distribution of the parameter, P(0), before running an analysis. Such priors may utilize existing domain knowledge to inform the model of likely values of the parameter (informative priors), or in situations wherein analysts have no, or do not wish to specify any, prior beliefs, non-informative priors may be utilized (e.g., Uniform(-infinity, infinity)). See for example Gelman et al, Bayesian Data Analysis, Third Edition, Chapman & Hall/CRC, 2013, London.

Regularization methods may be utilized in some embodiments of the invention as variable selection techniques and/or to improve predictive capabilities in models with a large number of parameters (p) and a (relatively) small number of data points (n). Such methods induce a penalization on the absolute magnitude of parameter estimates in models, and in some cases, drive estimates to zero and thereby providing automatic variable selection. Penalization methods include, without limitation, lasso, ridge, elastic net regression, or Bayesian methods with appropriately chosen priors to penalize parameter estimates towards the null value. See for example Casella G et al. Penalized regression, standard errors, and Bayesian lassos. (2010). Bayesian Analysis. 5, No. 2 pp 369-412, incorporated herein for reference.

In some embodiments of the present invention, decision trees are used to classify patients as having DOR or POI using genetic markers in combination with clinical metrics. Decision tree algorithms belong to the class of supervised learning algorithms. The aim of a decision tree is to induce a classifier (a tree) from real-world example data. This tree can then be used to classify unseen examples which have not been used to derive the decision tree.

In some embodiments of the invention, decision trees may be used to uncover how genetic markers and/or clinical metrics interact and increase the likelihood a patient is suffering from a disorder relating to ovarian reserve.

A decision tree is derived from training data. An example contains values for the different attributes and what class the example belongs. In one embodiment, the training data is data representative of a plurality of ovarian-reserve-associated phenotypic traits and genetic markers. The following algorithm describes a decision tree derivation:
Tree(Examples,Class,Attributes)
Create a root node
If all Examples have the same Class value, give the root this label
Else if Attributes is empty label the root according to the most common value
Else begin
Calculate the information gain for each attribute
Select the attribute A with highest information gain and make this the root attribute
For each possible value, v, of this attribute
Add a new branch below the root, corresponding to A=v
Let Examples(v) be those examples with A=v
If Examples(v) is empty, make the new branch a leaf node labeled with the most common value among Examples
Else let the new branch be the tree created by Tree(Examples(v),Class,Attributes-{A})
End

A more detailed description of the calculation of information gain is shown in the following. If the possible classes vᵢ of the examples have probabilities P(vᵢ) then the information content I of the actual answer is given by:

I(P(v₁), . . . ,P(vₙ))=nΣi=1−P(vᵢ)log 2P(vᵢ)

The I-value shows how much information we need in order to be able to describe the outcome of a classification for the specific dataset used. Supposing that the dataset contains p positive (e.g. DOR) and n negative (e.g. non-DOR) examples (e.g. individuals), the information contained in a correct answer is:

I(p/p+n,n/p+n)=−p/p+n log 2p/p+n−n/p+n log 2n/p+n

where log 2 is the logarithm using base two. By testing single attributes, the amount of information needed to make a correct classification can be reduced. The remainder for a specific attribute A (e.g. a trait) shows how much the information that is needed can be reduced.

Remainder(A)=vΣi=1pᵢ+nᵢ/p+nI(pᵢ/pᵢ+nᵢ,nᵢ/pᵢ+nᵢ)

“v” is the number of unique attribute values for attribute A in a certain dataset, “i” is a certain attribute value, “pᵢ” is the number of examples for attribute A where the classification is positive (e.g. DOR patient), “nᵢ” is the number of examples for attribute A where the classification is negative (e.g., non-DOR patient). The information gain of a specific attribute A is calculated as the difference between the information content for the classes and the remainder of attribute A:

Gain(A)=I(p/p+n,n/p+n)−Remainder(A)

The information gained is used to evaluate how important the different attributes are for the classification (how well they split up the examples), and the attribute with the highest information.

Decision tree algorithms often require consideration of feature processing, impurity measure, stopping criterion, and pruning. Specific decision tree algorithms include, cut are not limited to classification and regression trees (CART), multivariate decision trees, ID3, and C4.5. See, for example, Duda, Pattern Classification, Second Edition, 2001, John Wiley & Sons, Inc.

In one approach, when an exemplary embodiment of a decision tree is used, the data representative of a plurality of traits associated with ovarian reserve across a training population is standardized to have mean zero and unit variance. The members of the training population are randomly divided into a training set and a test set.

For example, in one embodiment, two-thirds of the members of the training population are placed in the training set and one third of the members of the training population are placed in the test set. The genetic markers and clinical metrics for a select combination of traits are used to construct the decision tree. Then, the ability for the decision tree to correctly classify members in the test set is determined. In some embodiments, this computation is performed several times for a given combination of genetic and clinical markers. In each iteration of the computation, the members of the training population are randomly assigned to the training set and the test set. Then, the quality of the combination of traits is taken as the average of each such iteration of the decision tree computation.

In some embodiments, the diagnosis-associated phenotypic traits and/or genotypic data are used to cluster a training set. For example, consider the case in which ten genetic variants described in the present invention are used. Each member m of the training population will have genotype values for each of the ten variants. Such values from a member m in the training population define the vector:
X1m X2m X3m X4m X5m X6m X7m X8m X9m X10m
   where Xim is the genotype of the i^{th} variant in organism m. If there are m organisms in the training set, selection of i variants will define m vectors. The methods of the present invention do not require that each possible variant of every single trait used in the vectors be represented in every single vector m. In other words, data from a subject in which one of the i^{th} traits is not found can still be used for clustering. In such instances, the missing expression value is assigned either a “zero” or some other normalized value. In some embodiments, prior to clustering, the trait expression values are normalized to have a mean value of zero and unit variance.

Those members of the training population that exhibit similar genotypes across the training group will tend to cluster together. A particular combination of traits of the present invention is considered to be a good classifier in this aspect of the invention when the vectors cluster into the trait groups found in the training population. For instance, if the training population includes patients with good or poor ovarian reserve, a clustering classifier will cluster the population into two groups, with each group uniquely representing either good or poor ovarian reserve. See, Duda and Hart, Pattern Classification and Scene Analysis, 1973, John Wiley & Sons, Inc., New York. The clustering problem is described as one of finding natural groupings in a dataset and to identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

One can begin a clustering analysis by using similarity measures, such as defining a distance function and to compute the matrix of distances between all pairs of samples in a dataset. When distance is a good measure of similarity, then the distance between samples in the same cluster will be significantly less than the distance between samples in different clusters. However, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. See, for example, a nonmetric similarity function s(x, x′) Duda, 216.

Once a method for measuring “similarity” or “dissimilarity” between points in a dataset has been selected, a criterion function that measures the clustering quality of any partition of the data must be identified. Partitions of the data set that extremize the criterion function are used to cluster the data. See Id. Duda. More recently, Duda et al., Pattern Classification, 2nd edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J.

Particular exemplary clustering techniques used in the present invention include, but are not limited to, hierarchical clustering (agglomerative clustering using nearest-neighbor algorithm, farthest-neighbor algorithm, the average linkage algorithm, the centroid algorithm, or the sum-of-squares algorithm), k-means clustering, fuzzy k-means clustering algorithm, and Jarvis-Patrick clustering.

Nearest neighbor classifiers are memory-based and require no model to be fit. Given a query point x0, the k training points x(r), r, . . . , k closest in distance to x0 are identified and then the point x0 is classified using the k nearest neighbors. Ties can be broken at random. In some embodiments, Euclidean distance in feature space is used to determine distance as:

d(i)=//x(i)−xo//.

Typically, when the nearest neighbor algorithm is used, the expression data used to compute the linear discriminant is standardized to have mean zero and variance 1. In the present invention, the members of the training population are randomly divided into a training set and a test set. For example, in one embodiment, two-thirds of the members of the training population are placed in the training set and one third of the members of the training population are placed in the test set. Profiles represent the feature space into which members of the test set are plotted. Next, the ability of the training set to correctly characterize the members of the test set is computed. In some embodiments, nearest neighbor computation is performed several times for a given combination of fertility-associated phenotypic traits. In each iteration of the computation, the members of the training population are randomly assigned to the training set and the test set. Then, the quality of the combination of traits is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis. See, Duda, Pattern Classification, Second Edition, 2001, John Wiley & Sons, Inc; and Hastie, 2001, The Elements of Statistical Learning, Springer, New York.

The pattern classification and statistical techniques for assessing ovarian reserve and function described above are merely examples of the types of models that can be used to construct a model for classification. It is to be understood that any statistical method can be used in accordance with the invention. Moreover, combinations of these described above also can be used.

The assessment of risk of ovarian dysfunction and/or premature decline in ovarian reserve of a female subject, which can be provided as probability of the subject having a disorder associated with declining ovarian reserve or function, such as DOR or POI, can be provided in the form of a report. The report can be generated and retrieved electronically and/or in paper format. The report can be used to provide information to the patient as well as to help guide treatment decisions of a physician.

### Treatment

In one embodiment, methods of targeting treatment upon assessment of risk of ovarian dysfunction and/or premature decline in ovarian reserve in a female subject are provided. For instance, with respect to POI, although most patients with POI experience complete infertility, early diagnosis of the disorder can indicate that that the patient may be able to achieve pregnancy and live birth by resorting to fertility treatments, including egg cryo-preservation, ovarian cortex cryo-preservation, and/or IVF before their conditions worsens. In other situations, a diagnosis of POI may indicate that pregnancy and live birth cannot be achieved using the female's own eggs, but can be achieved by IVF procedures using a donor egg(s). With respect to DOR, the patient may be able to achieve pregnancy and live birth by various treatment options such as, for example and not limitation, supplementation with the androgen dehydroepiandrosterone (DHEA), IVF, other fertility treatments known in the art, and combinations thereof. Additionally, treatment of DOR and/or POI can include treatment with immune function modulating therapies known in the art, such as TNF-inhibitors. Treatment of DOR and/or POI also includes treatment targeting inflammation, such as surgical and pharmacological interventions known in the art. It is also to be understood that treatment of DOR and/or POI, in accordance with the present invention, includes any other method known in the art now or that will eventually be developed that treats or prevents ovarian dysfunction and/or premature decline in ovarian reserve.

Fertility treatments in accordance with the present invention include, but are not limited to, assisted reproductive technologies (ART), non-ART fertility treatments (RE), and fertility preservation technologies (egg, embryo, or ovarian preservation). Exemplary assisted reproductive technologies include, without limitation, in vitro fertilization (IVF), zygote intrafallopian transfer (ZIFT), gametic intrafallopian transfer (GIFT), or intracytoplasmic sperm injection (ICSI) paired with one of the methods above. Exemplary non-ART fertility treatments include ovulation induction protocols with drugs such as Clomiphene or hormone therapy with or without intrauterine insemination (IUI) with sperm. In IVF, eggs are removed from the female subject, fertilized outside the body, and implanted inside the uterus of the female subject. ZIFT is similar to IVF in that eggs are removed and fertilization of the eggs occurs outside the body. In ZIFT, however, the eggs are implanted in the Fallopian tube rather than the uterus. GIFT involves transferring eggs and sperm into the female subject's Fallopian tube. Accordingly, fertilization occurs inside the woman's body. In ICSI, a single sperm is injected into a mature egg that has removed from the body. The embryo is then transferred to the uterus or Fallopian tube. In RE, hormone stimulation is used to improve the woman's fertility. Exemplary fertility preservation treatments include egg freezing in which eggs are removed, vitrified or otherwise frozen, and then stored indefinitely. Preservation can similarly be achieved through cryo-preservation of embryos generated through IVF and cryo-preservation of ovarian tissue, including slices of the ovarian cortex. Preservation could also involve removal of the ovary from the pelvic region and subcutaneous implantation in an ectopic location such as under the skin the in periphery of the body (i.e. arm).

### Systems

Aspects of the invention described herein can be performed using any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Methods of the invention can be performed using software, hardware, firmware, hardwiring, or combinations of any of these. Features implementing functions can also be physically located at various positions, including being distributed such that portions of functions are implemented at different physical locations (e.g., imaging apparatus in one room and host workstation in another, or in separate buildings, for example, with wireless or wired connections).

Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. For example, the reference set of data may be stored at a remote location and the computer communicates across a network to access the reference set to compare data derived from the female subject to the reference set. In other embodiments, however, the reference set is stored locally within the computer and the computer accesses the reference set within the CPU to compare subject data to the reference set. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTML5, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

In an exemplary embodiment shown in FIG. 14, system 401 can include a computer 433 (e.g., laptop, desktop, or tablet). The computer 433 may be configured to communicate across a network 415. Computer 433 includes one or more processor and memory as well as an input/output mechanism. Where methods of the invention employ a client/server architecture, any steps of methods of the invention may be performed using server 409, which includes one or more of processor and memory, capable of obtaining data, instructions, etc., or providing results via interface module or providing results as a file. Server 409 may be engaged over network 415 through computer 433 or terminal 467, or server 415 may be directly connected to terminal 467, including one or more processor and memory, as well as input/output mechanism. In some embodiments, systems include an instrument 455 for obtaining sequencing data, which may be coupled to a sequencer computer 451 for initial processing of sequence reads

Memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

Other embodiments are within the scope and spirit of the invention. For example, due to the nature of software, functions described above can be implemented using software, hardware, firmware, hardwiring, or combinations of any of these. Features implementing functions can also be physically located at various positions, including being distributed such that portions of functions are implemented at different physical locations.

### Example 1

In this example, whole genome sequencing was used to genetic factors underlying POI and DOR.

### Study Design and Methodology

The study subjects consisted of 4 women with POI, 12 women with DOR, and 36 women undergoing fertility treatment with evidence of normal ovarian reserve. All of the women were diagnosed and/or sought fertility treatment before the age of 38 and had clinical metrics consistent with their respective conditions, as shown below in Table 3.

TABLE 3
Patient Characteristics

| | Number of | | AMH | FSH | |
|---|---|---|---|---|---|
| | patients (N) | Age | (ng/ml) | (mIU/ml) | bAFC |
| DOR | 12 | 29 | 0.58 | 13.1 | 6.3 |
| POI | 4 | 32 | <0.16 | 48.9 | 1.7 |
| Normal | 36 | 32 | NA | 6.60 | 14 |
| Ovarian | | | | | |
| Reserve | | | | | |
| p-value | NA | 0.16 | 0.039 | 0.022 | 0.034 |

p-value refers to differences between POI and DOR and was calculated using the Wilcoxon test.
P-values <0.05 were considered significant.
AMH = anti-Mullerian hormone
FSH = follicle-stimulation hormone
bAFC = basal antral follicle count

Whole blood samples were taken from each of the study subjects. Genomic DNA was extracted from the whole blood. Whole genome sequences (with an average read depth of 30×) were generated using Illumina HiSeq sequencers. The sequences generated were then analyzed using GATK standard methods. Deleterious single nucleotide polymorphisms (SNPs) were identified using SNPeff, a variant effect prediction tool. A fertility-centric bioinformatics pipeline that incorporates pathway analysis tools was used to filter the SNPs. The Database for Annotation, Visualization and Integrated Discovery (DAVID) pathway analysis tool was used for gene annotation into functional pathways.

### Results

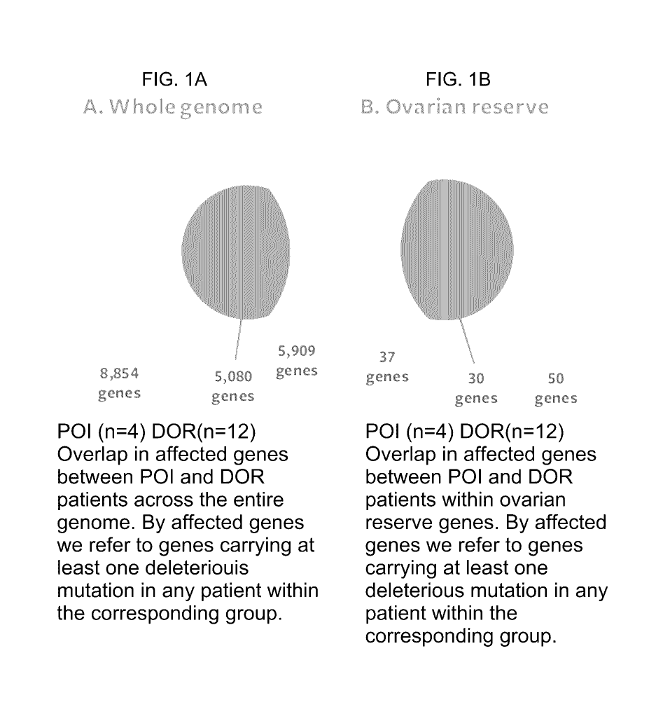

As shown in FIG. 1, a comparison of genes carrying at least one deleterious mutation in the POI and DOR groups uncovered significant amount of overlap in the affected genes in both patient groups. As can be seen, at the whole genome level, 5,909 and 8,854 genes were found to carry at least one deleterious mutation in the POI and DOR groups, respectively, and 5,080 genes were affected in both groups, as shown in FIG. 1A. Focusing on a curated list of ovarian reserve genes, 37 genes were found to be affected in POI patients, 50 genes were found to be affected in DOR patients and 30 genes were found to be affected in both, as shown in FIG. 1B.

Upon functional annotation of the curated ovarian reserve genes, it was found that most genes clustered in 8 functional processes, or categories, as shown in Table 4.

Focusing on gene variants within each functional category, their allele frequencies in the POI and DOR groups was compared to two control groups: a “normal ovarian reserve” group and the general population (1,000 genomes). As highlighted in Table 2, most gene variants that had significantly higher frequencies in POI or DOR patients compared to the control groups clustered in two functional categories: ovarian development and function and DNA repair.

As shown in FIG. 2A and in Table 5 below, five variants within the ovarian development and function pathway occurred at a significantly higher frequency in the POI cohort compared to the general population and the “normal ovarian reserve” group. See Wood et al., Genomic markers of ovarian reserve. Semin Reprod Med, 2013, 13(6):399-415. Additionally, four different variants within the ovarian development and function pathway were significantly more represented in the DOR group compared to the two control groups. Furthermore, variants in BMP15 and FSHR were exclusively found in the POI and DOR groups, respectively. These variants were not found in any patients within the “normal reserve” group.

TABLE 5
Variants within the ovarian development and function
pathway significantly represented in DOR or POI

| | |
|---|---|
| Gene | Variant(s) |

POI

| | |
|---|---|
| BMP15 | c.538G > A |
| NR5A1 | c.614C > T |
| FOXL2 | c.614C > T |
| KDR | c.1444T > C |
| | c.1082A > G |

DOR

| | |
|---|---|
| FSHR | c.485G > A |
| SHBG | c.473C > T |
| WNT4 | c.149-1G > C |
| FOXO3 | c.1037G > C |

Additionally, as shown in FIG. 2B and in Table 6 below, genes within the DNA repair pathway were also differentially affected in the POI and DOR groups, with the exception of FANCA, which was affected in both groups.

TABLE 6
Variants within the DNA repair pathway
significantly represented in DOR and/or POI

| | |
|---|---|
| Gene | Variant(s) |

POI

| | |
|---|---|
| BRCA1 | c.2174T > G |
| | c.662A > G |
| FANCA | c.3263C > T |
| | c.1235C > T |

DOR

| | |
|---|---|
| NBN | c.37G > A |
| POLG | c.3708G > T |
| MCM8 | c.1021G > A |
| FANCA | c.17T > A |
| | c.3263C > T |

As shown in FIG. 3, BMP15 is produced by the oocyte and is secreted into the follicular fluid (FIG. 3A). BMP15 also downregulates FSHR expression in these cells by decreasing their responsiveness to FSH (FIG. 3B). See., e.g., Otsuka F. et al., Bone Morphogenetic protein-15 inhibits follicle-stimulating hormone (FSH) action by suppressing FSH receptor expression. J. Biol. Chem, 2001, 276: 11387-11392. As found in POI patients, the BMP15 mutation leads to an amino acid change from alanine to threonine at position 180 in the pro-region of BMP15, as shown in FIG. 3A. This mutation was previously associated with POI and was believed to alter BMP15 dimerization and secretion and hence its ability to act on GCs. See, e.g., Laissue P. et al., Mutations and sequence variants in GDF9 and BMP15 in patients with premature ovarian failure. Eur J. Endocrinol 2006, 154: 739-744; Dixit H. et al., Missense mutations in the BMP15 gene are associated with ovarian failure. Hum Genet, 2006, 119:408-415.

As shown in FIG. 3C, the FSHR mutation detected in the DOR group leads to an amino acid change at position 162 from arginine to a lysine in the FSH binding region of the FSHR ectodomain. It is believed that this mutation alters the FSHR-FSH interaction.

As depicted by the results, although the ovarian reserve genes that were altered in the POI and DOR groups were different, they belonged to the same two functional pathways and in some cases affect similar biological processes. It was also found that BMP15 and FSHR are among genes uniquely affected in POI and DOR patients, respectively. Despite each gene uniquely affecting a different group of patients, the genes appear to alter the same biological process and decrease GCs responsiveness to FSH. Therefore, it is likely that POI and DOR are driven by similar etiologies.

The mutations identified using methods of the invention can be used as biomarkers for declining ovarian reserve and function, and ultimately fecundity, and can also be used to guide course of treatment.

Ongoing research is exploring whether the type of variants detected in the two patient groups impact the level of severity of the two conditions or whether genetic differences outside of the ovarian reserve gene network shed light on potential differences.

### Example 2

In this study, biological samples from 364 women (120 DOR/POI patients; 244 control patients) were analyzed to assess genetic correlates with a diagnosis of DOR or POI. It was hypothesized that eight genetic variants in the following genes, IL1A, IL18, TNF (3 variants), INHA, ICAM1, and GDF9 interact together, along with the woman's age, to contribute to an increased likelihood of DOR or POI.

The 28 possible pairwise interactions between genetic variants were assessed, along with an interaction between age and each genetic variant pair. The sample's genotype was treated numerically, corresponding to an additive genetic model.

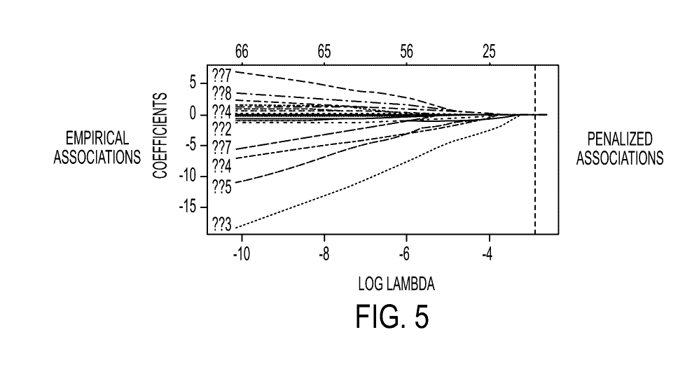

Least absolute shrinkage and selection operator (lasso) logistic regression was performed as the variable-selection technique to identify the variant interactions. Ten-fold cross validation was then performed to select the optimal penalization parameter (See FIG. 5). Surprisingly, the results obtained from the lasso model indicated a three-way interaction between age (mean-centered), ILIA, and GDF9 (Table 7), such that a joint-effect between all three of these variables in predicting the probability of a subject having a disorder associated with declining ovarian reserve or function (e.g., DOR or POI) is likely. All other parameter estimates in the model were regularized to zero (i.e., no evidence of an association).

TABLE 7
Non-zero regularized coefficients from lasso selection

| Coefficients | Penalized Logₑ (OR) |
|---|---|
| Age* | 0.00135 |
| Age:GDF9** | 0.0014 |
| Age:IL1A:GDF9*** | 0.0140 |

Next, the variability of the estimated effects of the three-way interaction between age, IL1A and GDF9 was assessed. Here, a penalized Bayesian logistic regression model was performed, fit to the three-way interaction between age and the IL1A and GDF9 variants. A horseshoe prior with three degrees of freedom was placed on the parameters of the model, corresponding to a penalized model. The posterior probability of the parameter for the three-way interaction being greater than zero (ie.g., evidence of an association) was 86.3%, suggesting that the joint effect of IL1A and GDF9 variant status on being diagnosed with DOR/POI differed as a function of age. The estimated odds ratios (ORs) for being diagnosed with DOR/POI relative to a patient with no risk alleles in either variant are shown in Tables 8(a) and 8(b) below, stratified by age. In Tables 8(a) and 8(b), d represents non-risk allele, D represents risk allele, and posterior probabilities of the OR being greater than 1 are included in parentheses. In Table 4(a) an analysis of women 35 years old is provided. The results show there is no evidence of a strong association between variant status in each variant and the odds of being diagnosed with DOR/POI for patients 35 years old. In Table 4(b) an analysis of women who are 42 years old is provided. Surprisingly, the results show that women who are 42 years old have an increasing number of risk alleles in each variant, which corresponds to increased odds of being diagnosed with DOR/POI.

TABLE 8(a)
Odds Ratio for Diagnosis of DOR/POI in 35 year old Women.

GDF9

| IL1A | dd | Dd | DD |
|---|---|---|---|
| dd | 1.0 (ref) | 0.98 (0.39) | 0.95 (0.39) |
| Dd | 1.03 (0.61) | 1.01 (0.51) | 0.98 (0.47) |
| DD | 1.06 (0.61) | 1.04 (0.54) | 1.01 (0.51) |

TABLE 8(b)
Odds Ratio for Diagnosis of DOR/POI in 42 year old Women.

GDF9

| IL1A | dd | Dd | DD |
|---|---|---|---|
| dd | 1.0 (ref) | 1.01 (0.53) | 1.02 (0.53) |
| Dd | 1.02 (0.57) | 1.06 (0.63) | 1.10 (0.62) |
| DD | 1.04 (0.57) | 1.11 (0.64) | 1.19 (0.63) |

As such, the likelihood of a woman over the age of 35 being diagnosed with DOR or POI is increased when variants in IL1A and GDF9 are present in her biological sample.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore

What is claimed is:

1. A method for assessing predicted ovarian reserve or function in a female subject, the method comprising using a computer system comprising a processor coupled to memory for:
accepting as input, data representative of a plurality of genetic and clinical characteristics of the female subject;
analyzing the input data using an ovarian reserve predictor correlated with ovarian reserve and function, wherein the ovarian reserve predictor was generated by:
obtaining reference data from a plurality of females, wherein the reference data corresponds to fertility and/or ovarian reserve-associated genetic and clinical characteristics and diagnoses of ovarian reserve dysfunction or decline;
determining one or more correlations between at least one genetic or clinical characteristic and a known diagnosis; and
generating a report of the probability of ovarian reserve dysfunction or decline in the female subject as a result of using the ovarian reserve predictor on the input data.

2. The method of claim 1, wherein the genetic characteristics are obtained by conducting an assay on a sample obtained from the female subject for the presence or absence of one or more variations associated with fertility and/or ovarian reserve or function.

3. The method of claim 2, wherein the assay comprises sequencing nucleic acid from the sample to determine the presence or absence of one or more mutations in one or more genes associated with fertility and/or ovarian reserve or function.

4. The method of claim 3, wherein the one or more genes are selected from the group consisting of: BMP15, FSHR, SHBG, FOXL2, KDR, NR5A1, WNT4, FOXO3, NBN, BRCA1, FANCA, MCM8, POLG, IL18, ICAM1, IL1A, INHA, GDF9, and TNF.

5. The method of claim 4, wherein the one or more genes are selected from the group consisting of: BMP15, INHA, IL1A, and GDF9.

6. The method of claim 2, wherein the assay comprises determining the expression level of one or more genes associated with fertility and/or ovarian reserve or function.

7. The method of claim 1, wherein the clinical characteristics are obtained from at least one selected from the group consisting of: analyzing a sample obtained from the female subject, a questionnaire, and a medical history of the subject.

8. The method of claim 7, wherein one or more of the clinical characteristics are selected from the group consisting of: age, basal antral follicle count (bARC), and anti-Mullerian hormone (AMH) levels.

9. The method of claim 1, wherein the known diagnosis comprises a diagnosis of primary ovarian insufficiency (POI) and/or diminished ovarian reserve (DOR).

10. A method for assessing an increased risk of ovarian reserve dysfunction or premature decline in a female subject, the method comprising the steps of:
obtaining a biological sample from the female subject;
isolating nucleic acid from said biological sample;
performing an assay on the isolated nucleic acid to determine a presence of one or more mutations in a gene, wherein the gene is associated with fertility and/or ovarian reserve or function;
assessing an increased risk of ovarian reserve dysfunction or premature decline based on the presence of one or more mutations in said gene, wherein the presence of at least one mutation in said gene is indicative of an increased risk of ovarian reserve dysfunction or premature decline in said female subject.

11. The method of claim 10, wherein the gene is selected from the group consisting of: BMP15, FSHR, SHBG, FOXL2, KDR, NR5A1, WNT4, FOXO3, NBN, BRCA1, FANCA, MCM8, POLG, IL18, ICAM1, IL1A, INHA, GDF9, and TNF.

12. The method of claim 11, wherein the gene is selected from the group consisting of BMP15, INHA, IL1A, and GDF9.

13. The method of claim 10, further comprising obtaining clinical characteristics from the female subject and assessing the increased risk based on the presence of the one or more variants and the clinical characteristics, wherein the clinical characteristics are associated with fertility and/or ovarian reserve or function.

14. The method of claim 13, wherein the clinical characteristics are obtained from at least one selected from the group consisting of: analyzing a sample obtained from the female subject, a questionnaire, and a medical history of the subject.

15. The method of claim 13, wherein one or more clinical characteristics are selected from the group consisting of: age, basal antral follicle count (bARC), and anti-Mullerian hormone (AMH) levels.

16. The method of claim 11, wherein the assay comprises sequencing the isolated nucleic acid to determine the presence or absence of one or more variants in a gene.

17. The method of claim 10, wherein the assay comprises determining the expression level of the gene.

18. A method of treating a female subject at risk for ovarian dysfunction or premature decline in ovarian reserve comprising:
conducting an assay to determine a presence of one or more variants in one or more genes associated with infertility and/or ovarian reserve or function, wherein the presence of the one or more variants is indicative that the female subject is more likely to suffer from a disorder associated with ovarian dysfunction or premature decline in ovarian reserve; and
providing a fertility treatment to the female subject based on the indicated disorder.

19. The method of claim 18, wherein the one or more genes are selected from the group consisting of: BMP15, INHA, IL1A, and GDF9.

20. The method of claim 18, wherein the indicated disorder is selected from the group consisting of primary ovarian insufficiency (POI) and diminished ovarian reserve (DOR).
